SEQUENCE LISTING (17 sequences)

SEQ ID NO: 1 - DNA, 30 bp, Artificial Sequence
  Primer used in conjunction with SEQ ID NO 2 to PCR-amplify a region of the Arabidopsis At7SL4 promoter
ggaattctgcgtttgaagaagagtgtttga

SEQ ID NO: 2 - DNA, 29 bp, Artificial Sequence
  Primer used in conjunction with SEQ ID NO 1 to PCR-amplify a region of the Arabidopsis At7SL4 promoter
gcccgggaagatcggttcgtgtaatatat

SEQ ID NO: 3 - DNA, 302 bp, Artificial Sequence
  Sequence of the PCR product amplified by SEQ ID NOs 1 and 2
ggaattctgcgtttgaagaagagtgtttgatgttctcaagtaagtgagtcttattgggaataatattaactcatgttcttcttgcatttgatttctttgccgctctcttcttctatctcaaatctgtctcttcaatttcacagttgggctttttattagtctataatgggactcaaaataaggctttggcccacatcaaaaagataagtcaaatgaaaactaaattcagtcttttgtcccacatcgatcactctactcgttttgtgtttgtttatatattacacgaaccgatcttcccgggc

SEQ ID NO: 4 - DNA, 37 bp, Artificial Sequence
  Primer used in conjunction with SEQ ID NO 5 to clone the 3′ NTS of the At7SL4 gene
gtctagattttgattttgttttccaaaactttctacg

SEQ ID NO: 5 - DNA, 28 bp, Artificial Sequence
  Primer used in conjunction with SEQ ID NO 4 to clone the 3′ NTS of the At7SL4 gene
gaagcttggtgttgatcacaacgataca

SEQ ID NO: 6 - DNA, 279 bp, Artificial Sequence
  Sequence of the 3′ NTS of the AtSL4 gene amplified by PCR using SEQ ID NOs 4 and 5
gtctagattttgattttgttttccaaaactttctacgctttttgtttttgggtttaatgctttaagagggaacaaaaacaaagctgtgaaaactgaaagcaaactttgaacaaagcaagagacttaagagttgtatttacagcttttgttcgatgtatggaaatgtacaatttttttgctactcaaagaaatgagacttaagagtcaacgttaaaagagccaggagtaaaatgtctaggtatgatctcaattgtatcgttgtgatcaacaccaagcttc

SEQ ID NO: 7 - DNA, 57 bp, Artificial Sequence
  Sense strand oligo that hybridizes to SEQ ID NO 8 to form a double stranded region
tacactgtggaattgatcagcgttcagatgacgctgatcaattccacagtttttttt

SEQ ID NO: 8 - DNA, 61 bp, Artificial Sequence
  Complementary oligo that hybridizes to SEQ ID NO 7 to form a double stranded region
ctagaaaaaaaactgtggaattgatcagcgtcatctgaacgctgatcaattccacagtgta

SEQ ID NO: 9 - DNA, 57 bp, Artificial Sequence
  Sense strand oligo that hybridizes to SEQ ID NO 10 to form a double stranded region
tacttgatcagcgttggtgggattcagatgatcccaccaacgctgatcaattttttt

SEQ ID NO: 10 - DNA, 61 bp, Artificial Sequence
  Complementary oligo that hybridizes to SEQ ID NO 9 to form a double stranded region
ctagaaaaaaattgatcagcgttggtgggatcatctgaatcccaccaacgctgatcaagta

SEQ ID NO: 11 - DNA, 57 bp, Artificial Sequence
  Sense strand oligo that hybridizes to SEQ ID NO 12 to form a double stranded region
tacctgtggaattgatcagcgtttcagatgaacgctgatcaattccacagttttttt

SEQ ID NO: 12 - DNA, 61 bp, Artificial Sequence
  Complementary oligo that hybridizes to SEQ ID NO 11 to form a double stranded region
ctagaaaaaaactgtggaattgatcagcgttcatctgaaacgctgatcaattccacaggta

SEQ ID NO: 13 - DNA, 1812 bp, Artificial Sequence
  Artificial GUS coding region
atgttacgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtacgttattgccgggaaaagtgtacgtatcaccgtttgtgtgaacaacgaactgaactggcagactatcccgccgggaatggtgactaccgacgaaaacggcaagaaaaagcagtcttacttccatgatttctttaactatgccggaatccatcgcagcgtaatgctctacaccacgccgagcacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgacgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgcaatgttctgcgacgttcacaccgataccatcagcgatctctttgatgtgctgtgcctgaaccgttattacggatggtatgtccaaagcggcgatttggaaacggcagagaaggtactggaaaaagaacttctggcctggcgggagaaactgcatcagccgattatcatcaccgaatacggcgtggatgcgttagccgggctgcactcaatgtacaccgacatgtggagtgaagagtatcagtgtgcatggctggatacgtatcaccgcgtctttgatcgcgtcagcgccgtcgtcggtgaacaggtatggagtttcgccgattttgcgacctcgcaaagcatattacgcgttggcggtagcaagaaagggatcttcactcgcgaccgcaaaccgaagtcggcggcttttctgctgcaaaaacgctggactggcataaacttcggtgaaaaaccgcagcagggaggcaaacaatga

SEQ ID NO: 14 - RNA, 19 nt, Artificial Sequence
  Sense strand of siRNA that corresponds to bases 80-98 of SEQ ID NO 13
acuguggaauugaucagcg

SEQ ID NO: 15 - RNA, 20 nt, Artificial Sequence
  Sequence of antisense strand that hybridizes to SEQ ID NO 14, plus 1 overhanging 3′ U
cgcugaucaauuccacaguu

SEQ ID NO: 16 - RNA, 19 nt, Artificial Sequence
  Sense strand of siRNA corresponding to bases 89-107 of SEQ ID NO 13
uugaucagcguugguggga

SEQ ID NO: 17 - RNA, 21 nt, Artificial Sequence
  Sequence of antisense strand that hybridizes to SEQ ID NO 16, plus 2 overhanging 3′ U′s
ucccaccaacgcugaucaauu

(19) United States
(12) Patent Application Publication
(10) Pub. No.: US 2005/0166289 A1
(43) Pub. Date: Jul. 28, 2005

(54) Small interfering RNA (siRNA)-mediated heritable gene manipulation in plants

(75) Inventors: Vincent Lee Chuan Chiang, Cary, NC (US); Shanfa Lu, Raleigh, NC (US); Laigeng Li, Cary, NC (US)

Correspondence Address: JENKINS, WILSON & TAYLOR, P. A., 3100 TOWER BLVD, SUITE 1400, DURHAM, NC, 27707, US

(73) Assignee: North Carolina State University, Raleigh, NC (US)

(21) Appl. No.: 11/000,863

(22) Filed: Dec. 1, 2004

Related U.S. Application Data
(60) Provisional application No. 60/526,127, filed on Dec. 1, 2003.
(60) Provisional application No. 60/537,461, filed on Jan. 16, 2004.

(51) Int. Cl. A01H 1/00; C12N 15/82

(52) U.S. Cl. 800/286

(57) ABSTRACT

The presently disclosed subject matter provides methods and compositions for stably modulating gene expression in plants. Also provided are plants and cells comprising the compositions of the presently disclosed subject matter.

### CROSS REFERENCE TO RELATED APPLICATIONS

This application is based on and claims priority to U.S. Provisional Application Ser. Nos. 60/526,127, filed Dec. 1, 2003, and 60/537,461, filed Jan. 16, 2004, the disclosures of which are herein incorporated by reference in their entireties.

### TECHNICAL FIELD

The presently disclosed subject matter relates, in general, to methods and compositions for modulating gene expression in a plant. More particularly, the presently disclosed subject matter relates to a method of using an siRNA-encoding binary vector to stably modulate the expression level of a gene in a plant.

Table of Abbreviations

| | |
|---|---|
| 2,4-D | 2,4-dichlorophenoxyacetic acid |
| 3′-NTS | 3′ non-transcribed sequence |
| AMV | Alfalfa Mosaic Virus |
| bp | basepair(s) |
| CMV | cytomegalovirus |
| DHFR | dihydrofolate reductase |
| dsRNA | double stranded RNA |
| EDTA | ethylenediamine tetraacetic acid |
| GUS | β-glucuronidase |
| GUS | a human β-glucuronidase gene or |
| | nucleotide sequence |
| H1-P | a nucleic acid molecule comprising the |
| | human H1 gene promoter |
| HPRT | hypoxanthine phosphoribosyl transferase |
| hpt | hygromycin phosphotransferase selectable |
| | marker gene |
| hsp | heat shock protein |
| HSPs | high scoring sequence pairs |
| MCMV | Maize Chlorotic Mottle Virus |
| MCS | multiple cloning site |
| nt | nucleotide(s) |
| ORF | open reading frame |
| PEG | polyethylene glycol |
| PEPC | phosphoenol carboxylase |
| PGK | phosphoglycerate kinase |
| PKR | RNA-dependent protein kinase |
| PTGS | post-transcriptional gene silencing |
| RISC | RNA-induced silencing complex |
| RNAi | RNA interference |
| siRNA(s) | small interfering RNA(s) |
| SDS | sodium dodecyl sulfate |
| SSC | standard saline citrate (0.15 M NaCl; 0.015 M |
| | sodium citrate, pH 7.0) |
| SV40 | simian virus 40 |
| T | thymidine or thymine |
| TAFs | transcription associated factors |
| TBE | tris-borate-EDTA |
| Tₘ | thermal melting point |
| TMV | Tobacco Mosaic Virus |
| U | uridine or uracil |
| USE | upstream sequence element |

Amino Acid Abbreviations and Corresponding mRNA Codons

| Amino Acid | 3-Letter | 1-Letter | mRNA Codons |
|---|---|---|---|
| Alanine | Ala | A | GCA; GCC; GCG; GCU |
| Arginine | Arg | R | AGA; AGG; CGA; CGC; |
| | | | CGG; CGU |
| Asparagine | Asn | N | AAC; AAU |
| Aspartic Acid | Asp | D | GAC; GAU |
| Cysteine | Cys | C | UGC; UGU |
| Glutamic Acid | Glu | E | GAA; GAG |
| Glutamine | Gln | Q | CAA; CAG |
| Glycine | Gly | G | GGA; GGC; GGG; GGU |
| Histidine | His | H | CAC; CAU |
| Isoleucine | Ile | I | AUA; AUC; AUU |
| Leucine | Leu | L | UUA; UUG; CUA; CUC; |
| | | | CUG; CUU |
| Lysine | Lys | K | AAA; AAG |
| Methionine | Met | M | AUG |
| Proline | Pro | P | CCA; CCC; CCG; CCU |
| Phenylalanine | Phe | F | UUC; UUU |
| Serine | Ser | S | ACG; AGU; UCA; UCC; |
| | | | UCG; UCU |
| Threonine | Thr | T | ACA; ACC; ACG; ACU |
| Tryptophan | Trp | W | UGG |
| Tyrosine | Tyr | Y | UAC; UAU |
| Valine | Val | V | GUA; GUC; GUG; GUU |

### BACKGROUND

A common defense mechanism in plants and mammals against viral infection involves the generation of small interfering RNAs (siRNAs) of about 21-23 nucleotides (nt) that can recognize specific sequences in the invading viral RNA genome to guide its destruction (Waterhouse et al., 2001; McManus & Sharp, 2002). This mechanism has led to the use of synthetic siRNAs in mammalian cells to induce transient silencing of specific target mRNAs as a tool for understanding gene function (McManus & Sharp, 2002; Dillin, 2003).

Systems for intracellular expression of siRNAs from plasmid DNA have also been developed recently for mammalian cells, allowing the analysis of loss-of-function phenotypes that arise over longer time periods (Brummelkamp et al., 2002; Lee et al., 2002; Miyagishi & Taira, 2002; Paddison et al., 2002; Paul et al., 2002; Sui et al., 2002; Yu et al., 2002). In these systems, the human RNA polymerase III U6 or H1 RNA gene promoters, which are known for processing small RNA transcripts, were used to induce the endogenous transcription of the target siRNAs. In one report, sense and antisense siDNA strands encoding a target siRNA duplex of 21 nucleotides were transcribed by individual U6 promoters (Miyagishi & Taira, 2002). In another report, siRNA sequences were transcribed by a single H1 promoter into a fold-back stem-loop structure that was predicted to give rise to an siRNA duplex of 19 nucleotides (Brummelkamp et al., 2002). In both reports, the siRNA duplex contained two to four 3′ overhanging uridine (U) nucleotides, resembling the structure of molecules that have been reported in other systems as being capable of initiating RNA interference for specific mRNA degradation (Elbashir et al., 2001a; Elbashir et al., 2001b.

Current approaches for elucidating gene function or for trait modification in plants are characterized by a lack of specificity. Additionally, stable siRNA-mediated gene silencing has as yet not been achieved in plants.

Thus, there exists a long-felt and continuing need in the art for effective strategies for specifically and stably modulating the expression of genes in plants. The presently disclosed subject matter addresses this and other needs in the art.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides a method for stably modulating expression of a plant gene. In one embodiment, the method comprises (a) providing a vector encoding a short interfering RNA (siRNA) targeted to the plant gene; and (b) transforming a plant with the vector, whereby stable expression of the siRNA in the plant is provided. In one embodiment, the vector is an Agrobacterium binary vector. In another embodiment, the vector comprises (a) a promoter operatively linked to a nucleic acid molecule encoding the siRNA molecule; and (b) a transcription termination sequence. In one embodiment, the promoter is a DNA-dependent RNA polymerase III promoter. In another embodiment, the promoter is selected from the group consisting of an RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, and a tRNA gene promoter, or functional derivatives thereof. In one embodiment, the Arabidopsis thaliana 7SL RNA gene promoter comprises the sequence presented in SEQ ID NO: 3.

In one embodiment of the present method, the nucleic acid sequence encoding the short interfering RNA (siRNA) molecule comprises a sense region, an antisense region, and a loop region, positioned in relation to each other such that upon transcription, the resulting RNA molecule is capable of forming a hairpin structure via intramolecular hybridization of the sense strand and the antisense strand.

The methods of the presently disclosed subject matter can be used to modulate gene expression in any plant. In one embodiment, the plant is a dicot. In another embodiment, the plant is a monocot. In another embodiment, the plant is a tree. In one embodiment, the tree is an angiosperm. In another embodiment, the tree is a gymnosperm. In still another embodiment, the plant is selected from the group consisting of Arabidopsis, poplar, aspen, and tobacco.

In one embodiment of the presently disclosed subject matter, stable expression of the short interfering RNA (siRNA) in the plant occurs in a location or tissue selected from the group consisting of epidermis, root, vascular tissue, xylem, meristem, cambium, cortex, pith, leaf, flower, seed, and combinations thereof.

The presently disclosed subject matter also provides a vector for stably expressing a short interfering RNA (siRNA) molecule in a plant. In one embodiment, the vector comprises (a) a promoter operatively linked to a nucleic acid molecule encoding the siRNA molecule; and (b) a transcription termination sequence. In one embodiment, the vector is an Agrobacterium binary vector. In one embodiment, the promoter is a DNA-dependent RNA polymerase III promoter. In another embodiment, the promoter is selected from the group consisting of RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, and a tRNA gene promoter, or a functional derivative thereof. In one embodiment, the Arabidopsis thaliana SL7 RNA gene promoter comprises the sequence presented in SEQ ID NO: 3. In one embodiment, the nucleic acid sequence encoding the short interfering RNA (siRNA) molecule comprises a sense region, an antisense region, and a loop region, positioned in relation to each other such that upon transcription, the resulting RNA molecule is capable of forming a hairpin structure via intramolecular hybridization of the sense strand and the antisense strand.

The presently disclosed subject matter also provides a kit comprising a disclosed vector and at least one reagent for introducing the disclosed vector into a plant cell. In one embodiment, the kit further comprises instructions for introducing the vector into a plant cell.

Also provided are plant cells and transgenic plants comprising the disclosed vectors, as well as transgenic seed or progeny from the disclosed transgenic plants.

The presently disclosed subject matter also provides a method for enhancing the expression of a gene in a plant cell. In one embodiment, the method comprises introducing into the plant cell a vector encoding a short interfering RNA (siRNA) molecule corresponding to at least a subsequence of the gene, wherein the gene is selected from the group consisting of coniferaldehyde-5-hydroxylase (Cald5H); lignin-related genes, including SAD, CAD, 4CL, CCoAOMT, COMT, F5H, C4H, C3H, CCR, and PAL; cellulose-related genes, including cellulose synthase, cellulose synthase-like, glucosidase, glucan synthase, and sucrose synthase; hormone-related genes, including ipt, GA oxidase, AUX, and ROL; disease-related genes; stress-related genes; and transcription factor genes. In another embodiment, the method comprises introducing into the plant cell a vector encoding a short interfering RNA (siRNA) molecule comprising a sequence that hybridizes to a nucleic acid molecule encoding a repressor of a gene, thereby resulting in downregulation of expression of the repressor.

Also provided is a method for stably inhibiting expression of a gene in a plant cell. In one embodiment, the method comprises introducing a vector encoding an siRNA into the cell in an amount sufficient to inhibit expression of the gene, wherein the siRNA comprises a ribonucleotide sequence that corresponds to at least 15 contiguous nucleotides of a coding strand of the gene. In representative embodiments of the present method, the gene is selected from the group consisting of lignin-related genes, including SAD, CAD, 4CL, CCoAOMT, COMT, F5H, C4H, C3H, CCR, and PAL; cellulose-related genes, including cellulose synthase, cellulose synthase-like, glucosidase, glucan synthase, and sucrose synthase; hormone-related genes, including ipt, GA oxidase, AUX, and ROL; disease-related genes; stress-related genes; and transcription factor genes. In one embodiment, the siRNA comprises a double-stranded region comprising a first strand comprising a ribonucleotide sequence that corresponds to a coding strand of the gene and a second strand comprising a ribonucleotide sequence that is complementary to the first strand, and wherein the first strand and the second strand hybridize to each other to form the double-stranded region. In one embodiment, the double stranded region is at least 15 basepairs in length. In another embodiment, the double stranded region is between 15 and 50 basepairs in length. In another embodiment, the double stranded region is between 15 and 30 basepairs in length. In another embodiment, the length of the double stranded region is selected from the group consisting of 19, 20, 21, 22, 23, 24, 25, and 26 basepairs. In still another embodiment, the length of the double stranded region is 19 basepairs. In one embodiment, the RNA comprises one strand that forms a double-stranded region of at least 19 basepairs by intramolecular self-hybridization. In one embodiment of the present method, the expression of the gene is inhibited by at least 10%.

Also provided is an expression vector encoding a short interfering RNA (siRNA) molecule that stably down regulates expression of a plant gene by RNA interference. In one embodiment, the short interfering RNA (siRNA) molecule comprises a sense region and an antisense region, and wherein the antisense region comprises a nucleic acid sequence complementary to an RNA sequence encoded by the plant gene and the sense region comprises a nucleic acid sequence complementary to the antisense region. In another embodiment, the short interfering RNA (siRNA) molecule is assembled from two nucleic acid fragments, wherein one fragment comprises a sense region and the other fragment comprises an antisense region of the siRNA molecule. In one embodiment, the sense region and antisense region are covalently connected via a linker molecule. In one embodiment, the linker molecule is a polynucleotide linker. In one embodiment, the polynucleotide linker comprises from 5 to 9 nucleotides.

In one embodiment of the present expression vector, the short interfering RNA (siRNA) molecule is formed by intramolecular self-hybridization of the sense region and the antisense region to produce a double-stranded molecule, and the double-stranded molecule comprises a 3′-terminal overhang of at least 1 nucleotide. In one embodiment, the 3′-terminal overhang comprises from 1 to 8 nucleotides. In one embodiment, the antisense region is complementary to a ribonucleic acid (RNA) transcribed from a gene selected from the group consisting of lignin-related genes, including SAD, CAD, 4CL, CCoAOMT, COMT, F5H, C4H, C3H, CCR, and PAL; cellulose-related genes, including cellulose synthase, cellulose synthase-like, glucosidase, glucan synthase, and sucrose synthase; hormone-related genes, including ipt, GA oxidase, AUX, and ROL; disease-related genes; stress-related genes; and transcription factor genes.

In another embodiment of the expression vector disclosed herein, the short interfering RNA (siRNA) molecule comprises a sense region and an antisense region and wherein the antisense region comprises a nucleic acid sequence complementary to an RNA sequence transcribed from a gene selected from the group consisting of lignin-related genes, including SAD, CAD, 4CL, CCoAOMT, COMT, F5H, C4H, C3H, CCR, and PAL; cellulose-related genes, including cellulose synthase, cellulose synthase-like, glucosidase, glucan synthase, and sucrose synthase; hormone-related genes, including ipt, GA oxidase, AUX, and ROL; disease-related genes; stress-related genes; and transcription factor genes, and the sense region comprises a nucleic acid sequence complementary to the antisense region. In another embodiment, the short interfering RNA (siRNA) molecule comprises a single strand having complementary sense and antisense regions.

The presently disclosed subject matter also provides a plant cell comprising an expression vector as disclosed herein. In one embodiment, the plant cell is from a plant selected from the group consisting of poplar, pine, eucalyptus, sweetgum, other tree species, tobacco, Arabidopsis, rice, corn, wheat, cotton, potato, and cucumber.

Also provided is a plasmid vector encoding a short interfering RNA (siRNA) molecule that stably down regulates expression of a plant gene by RNA interference. In one embodiment, the short interfering RNA (siRNA) molecule comprises a sense region and an antisense region, and wherein the antisense region comprises a nucleic acid sequence complementary to an RNA sequence encoded by the plant gene and the sense region comprises a nucleic acid sequence complementary to the antisense region.

The presently disclosed subject matter also provides a vector for the stable expression of a short interfering RNA (siRNA) in a plant, wherein the vector comprises a promoter for expressing the siRNA, a transcription termination sequence, and a cloning site between the promoter and the transcription termination sequence into which a nucleic acid molecule encoding the siRNA can be cloned. In one embodiment, the promoter is a DNA-dependent RNA polymerase III promoter. In representative embodiments, the promoter is selected from the group consisting of RNA polymerase III H1 promoter, an RNA polymerase III 7SL promoter, an RNA polymerase III 5S promoter, an RNA polymerase III U6 promoter, an adenovirus VA1 promoter, a Vault promoter, a telomerase RNA promoter, and a tRNA gene promoter, or a functional derivative thereof. In one embodiment, the Arabidopsis thaliana 7SL RNA gene promoter comprises SEQ ID NO: 3. In one embodiment, the vector is a plasmid vector.

In another embodiment of the instant vector, the vector further comprises a selectable marker. In one embodiment, the vector further comprises a cloning site comprising recognition sequences for at least two restriction enzymes that are not present elsewhere in the plasmid vector.

The presently disclosed subject matter also provides a method for stably modulating expression of a gene in a plant comprising (a) transforming a plurality of plant cells to create a plurality of transformed plant cells, wherein the transformed plants cells have been transformed with a vector comprising a nucleic acid sequence encoding a short interfering RNA (siRNA) operatively linked to a promoter and a transcription termination sequence; (b) growing the transformed plant cells under conditions sufficient to select for those transformed plant cells that have integrated the vector into their genomes; (c) screening the plurality of transformed plant cells for expression of the siRNA encoded by the vector; (d) selecting a plant cell that expresses the siRNA; and (e) regenerating the plant from the plant cell that expresses the siRNA, whereby expression of the gene in the plant is stably modulated. In one embodiment, the nucleic acid sequence encoding the short interfering RNA (siRNA) comprises (a) a sense region;(b) an antisense region; and (c) a loop region, wherein the sense, antisense, and loop regions are positioned in relation to each other such that upon transcription, the resulting RNA molecule is capable of forming a hairpin structure via intramolecular hybridization of the sense strand and the antisense strand. In one embodiment, the vector is an Agrobacterium binary vector further comprising a nucleic acid encoding a selectable marker operatively linked to a promoter.

Accordingly, it is an object of the presently disclosed subject matter to provide a method for stably manipulating gene expression in plants using an siRNA-mediated approach. This object is achieved in whole or in part by the presently disclosed subject matter.

An object of the presently disclosed subject matter having been stated above, other objects and advantages will become apparent to those of ordinary skill in the art after a study of the following description of the presently disclosed subject matter and non-limiting Examples.

### BRIEF DESCRIPTION OF THE DRAWINGS

FIGS. 1A-1E depict human H1 promoter-mediated siRNA silencing of GUS gene expression in transgenic tobacco.

FIG. 1A depicts GUS staining of cross-sections of the stems, of the leaves, and of the roots of one month old siRNA-transgenic (GT1 and GT2) and GUS-expressing control (C) tobacco plants.

FIG. 1B is a graph of GUS protein activity (Jefferson et al., 1987) in the leaves of control plants and of ten GT2 transgenic plants. Mean values were calculated from three independent measurements per line.

FIG. 1C depicts a loading control for gel blot analysis of RNA transcript level using a 25S ribosomal RNA probe.

FIG. 1D depicts the same gel blot as shown in FIG. 1C, but is used to characterize the level of GUS mRNA using a GUS cDNA probe.

FIG. 1E depicts gel blot detection of siRNAs of about 21 nt (position indicated) using a GUS cDNA probe as described in Hutvagner et al., 2000. RNA was isolated from a portion of the leaves used for the GUS protein activity assay depicted in FIG. 1B.

FIG. 2 depicts a schematic representation of plasmid pUCSL1. The plasmid contains a promoter fragment (289 basepairs; P_{7SL-RNA}) containing USE and TATA elements and a 3′-NTS fragment (267 basepairs) from the Arabidopsis thaliana At7SL4 gene, cloned into pUC19. Between the promoter and 3′-NTS sequences is a multiple cloning site (MCS) containing recognition sequences for SmaI, BamHI, and XbaI, which can be used to clone siRNA sequences. The promoter:MCS:3′-NTS cassette can be excised from pUCSL1 using EcoRI and HindIII sites which are present at the 5′ and 3′ ends of the cassette, respectively.

FIG. 3 depicts a schematic representation of plasmid pSIT. The plasmid contains the promoter:MCS:3′-NTS cassette from pUCSL1 in the opposite transcriptional orientation and downstream of a selectable marker cassette, the latter consisting of a promoter, selectable marker gene, and terminator sequence. pSIT represents a binary vector transformation system mediated by Agrobacterium.

FIG. 4 depicts a representation of the multiple cloning site (MCS) of pSIT. Between the SmaI and XbaI sites of the MCS is cloned a sequence comprising 19-26 nt from the sense strand of the gene of interest, followed by a 9 nt spacer, and then the reverse complement of the 19-26 nt sequence (i.e., the antisense sequence cloned in the opposite direction). Downstream of the antisense sequence is the sequence TTTTTTT, which serves to terminate transcription from the promoter for siRNA transcription present in pSIT (see FIG. 3).

FIG. 5 depicts the preparation of siRNA expression constructs. The 19 nucleotide (nt) GUS gene-specific sequence (GT1 represented nucleotide positions 80-98 and GT2 89-107) separated by a 9 nt spacer from the reverse complement of the same sequence followed by a termination signal of five thymidines was cloned into pSUPER (available from OligoEngine, Inc., Seattle, Wash., United States of America) downstream of the H1 promoter (H1-P). The H1-P::GT expression construct was then excised and cloned into the binary vector pGPTV-HPT (Becker et al., 1992) to replace the pAnos-uidA fragment. The resulting vector, pGPH1-HPT, which contained a hygromycin phosphotransferase selectable marker gene (hpt), was then mobilized into Agrobacterium tumefaciens C58 for transforming tobacco. The predicted secondary siRNA structures of GT1 and GT2 are depicted at the bottom of the Figure. Considered in the 5′ to 3′ direction, FIG. 5 shows the sequences of GT1 and GT2 that form the hairpin as follows. For GT1, the hairpin is produced by the intramolecular hybridization of SEQ ID NO: 14 and SEQ ID NO: 15, with a 9 nt spacer between. For GT2, the hairpin is produced by the intramolecular hybridization of SEQ ID NO: 16 and SEQ ID NO: 17, with a 9 nt spacer between. FIG. 5 depicts these hairpins with the “top” strand in the 5′ to 3′ direction, and thus the “bottom” strand is depicted in the 3′ to 5′ direction.

FIGS. 6A-6E depict plant 7SL promoter-mediated siRNA silencing of GUS gene expression in transgenic tobacco.

FIG. 6A depicts an Agrobacterium binary vector for the expression of GUS-specific hairpin siRNAs. The abbreviations presented in FIG. 6A are as follows: 7SL-P: a promoter fragment (289 bp) containing USE and TATA elements of the Arabidopsis At7SL49 gene; UT: a 267 basepair region of the 3′ untranslated region of the Arabidopsis At7SL49 gene; Pnos: nopaline synthase promoter; hpt: a hygromycin phosphotransferase selectable marker coding sequence; pAg7: an agropine synthase polyadenylation signal sequence.

FIG. 6B presents GUS protein activity in leaves of the control (C) plants and eleven GT2 transgenics. Mean values were calculated from three independent measurements per line.

FIG. 6C depicts an RNA loading control.

FIG. 6D depicts the same gel blot used in FIG. 6C, which was also used to characterize GUS mRNA levels using a GUS cDNA probe.

FIG. 6E depicts gel blot detection of small RNAs of about 21-nt using a GUS cDNA probe.

### BRIEF DESCRIPTION OF THE SEQUENCE LISTING

SEQ ID NOS: 1 and 2 are primer sequences used to PCR-amplify a region of the Arabidopsis At7SL4 promoter.

SEQ ID NO: 3 is the nucleic acid sequence of the product of a PCR reaction using the primers identified in SEQ ID NOS: 1 and 2.

SEQ ID NOS: 4 and 5 are primer used to amplify the 3′-NTS of the At7SL4 gene.

SEQ ID NO: 6 is the nucleic acid sequence of the product of a PCR reaction using the primers identified in SEQ ID NOS: 4 and 5.

SEQ ID NOS: 7-12 are the sequences of complementary oligonucleotides that were used to generate siRNAs targeted to the GUS gene. Three different regions of the GUS gene were targeted. For the production of pGSGT1, SEQ ID NOS: 7 and 8 were hybridized to each other. For the production of pGSGT2, SEQ ID NOS: 9 and 10 were hybridized to each other. For the production of pGSGT3, SEQ ID NOS: 11 and 12 were hybridized to each other.

SEQ ID NO: 13 is the nucleic acid sequence of an artificial GUS open reading frame (GENBANK® Accession No. AY100472).

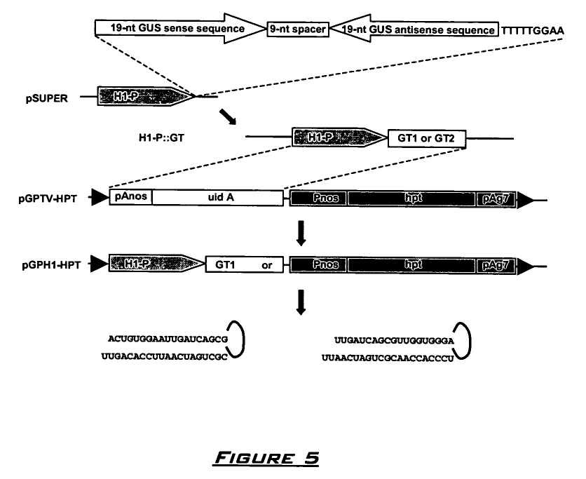

SEQ ID NOS: 14-17 are presented in FIG. 5, and correspond to the sense and antisense sequences for representative siRNA-like molecules targeting the GUS gene. SEQ ID NO: 14 is a nucleic acid sequence that corresponds to bases 80-98 of SEQ ID NO: 13, and is a sense strand sequence. SEQ ID NO: 15 is a nucleic acid sequence that hybridizes to SEQ ID NO: 14 and includes a one nucleotide 3′ overhang (U). SEQ ID NO: 16 is a nucleic acid sequence that corresponds to bases 89-107 of SEQ ID NO: 13, and is a sense strand sequence. SEQ ID NO: 17 is a nucleic acid sequence that hybridizes to SEQ ID NO: 16 and includes a two nucleotide 3′ overhangs (UU).

### DETAILED DESCRIPTION

I. General Considerations

The approach to gene function characterization through the use of small interfering RNAs (siRNAs) offers the potential for agriculture and tree crop improvement. However, in plant systems, siRNA-mediated interference of gene expression has only been possible at the transient level. Described herein are DNA plasmid-based siRNA expression systems for the heritable modulation of gene expression (for example, using Agrobacterium-mediated transformation). These systems allow the application of a potent siRNA-based approach to trait modification as well as gene function analysis in plants.

The presently disclosed subject matter takes advantage of the ability of short, double stranded RNA molecules to modulate the expression of cellular genes, a process referred to as RNA interference (RNAi) or post transcriptional gene silencing (PTGS). As used herein, the terms “RNA interference” and “post-transcriptional gene silencing” are used interchangeably and refer to a process of sequence-specific downregulation of gene expression mediated by a small interfering RNA (siRNA). See generally Fire et al., 1998. The process of post-transcriptional gene silencing is thought to be an evolutionarily conserved cellular defense mechanism that has evolved to prevent the expression of foreign genes (Fire, 1999).

RNAi might have evolved to protect cells and organisms against the production of double stranded RNA (dsRNA) molecules resulting from infection by certain viruses (particularly the double stranded RNA viruses or those viruses for which the life cycle includes a double stranded RNA intermediate) or the random integration of transposon elements into the host genome via a mechanism that specifically degrades single stranded RNA or viral genomic RNA homologous to the double stranded RNA species.

The presence of dsRNA in cells triggers various responses, one of which is RNAi. RNAi appears to be different from the interferon response to dsRNA, which results from dsRNA-mediated activation of an RNA-dependent protein kinase (PKR) and 2′,5′-oligoadenylate synthetase, resulting in non-specific cleavage of mRNA by ribonuclease L.

The presence of long dsRNAs in plant or animal cells stimulates the activity of the enzyme Dicer, a ribonuclease III. Dicer catalyzes the degradation of dsRNA into short stretches of dsRNA referred to as small interfering RNAs (siRNA; Bernstein et al., 2001). The small interfering RNAs that result from Dicer-mediated degradation are typically about 21-23 nucleotides in length and contain about 19 base pair duplexes. After degradation, the siRNA is incorporated into an endonuclease complex referred to as an RNA-induced silencing complex (RISC). The RISC is capable of mediating cleavage of single stranded RNA present within the cell that is complementary to the antisense strand of the siRNA duplex. According to Elbashir et al., cleavage of the target RNA occurs near the middle of the region of the single stranded RNA that is complementary to the antisense strand of the siRNA duplex (Elbashir et al., 2001 b).

RNAi has been described in several cell type and organisms. Fire et al., 1998 described RNAi in C. elegans. Wianny & Zernicka-Goetz, 1999 disclose RNAi mediated by dsRNA in mouse embryos. Hammond et al., 2000 were able to induce RNAi in Drosophila cells by transfecting dsRNA into these cells. Elbashir et al., 2001a demonstrated the presence of RNAi in cultured mammalian cells including human embryonic kidney and HeLa cells by the introduction of duplexes of synthetic 21 nucleotide RNAs.

Experiments using Drosophila embryonic lysates revealed certain aspects of siRNA length, structure, chemical composition, and sequence that are involved in RNAi activity. See Elbashir et al., 2001c. In this assay, 21 nucleotide siRNA duplexes were most active when they contain 3′-overhangs of two nucleotides. Also, the position of the cleavage site in the target RNA was shown to be defined by the 5′-end of the siRNA guide sequence rather than the 3′-end (Elbashir et al., 2001 b).

Other studies have indicated that a 5′-phosphate on the target-complementary strand of a siRNA duplex is required for siRNA activity and that ATP is utilized to maintain the 5′-phosphate moiety on the siRNA (Nykanen et al., 2001). Other modifications that might be tolerated when introduced into an siRNA molecule include modifications of the sugar-phosphate backbone or the substitution of the nucleoside with at least one of a nitrogen or sulfur heteroatom (PCT International Publication Nos. WO 00/44914 and WO 01/68836) and certain nucleotide modifications that might inhibit the activation of double stranded RNA-dependent protein kinase (PKR), specifically 2′-amino or 2′-O-methyl nucleotides, and nucleotides containing a 2′-O or 4′-C methylene bridge (Canadian Patent Application No. 2,359,180).

Other references disclosing the use of dsRNA and RNAi include PCT International Publication Nos. WO 01/75164 (in vitro RNAi system using cells from Drosophila and the use of specific siRNA molecules for certain functional genomic and certain therapeutic applications); WO 01/36646 (methods for inhibiting the expression of particular genes in mammalian cells using dsRNA molecules); WO 99/32619 (methods for introducing dsRNA molecules into cells for use in inhibiting gene expression); WO 01/92513 (methods for mediating gene suppression by using factors that enhance RNAi); WO 02/44321 (synthetic siRNA constructs); WO 00/63364 and WO 01/04313 (methods and compositions for inhibiting the function of polynucleotide sequences); and WO 02/055692 and WO 02/055693 (methods for inhibiting gene expression using RNAi).

II. Definitions

For convenience, certain terms employed in the specification, examples, and appended claims are collected here. While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in this application, including the claims. Thus, the articles “a”, “an”, and “the” are used herein to refer to one or more than one (i.e., to at least one) of the grammatical object of the article unless the context in which the article appears clearly demonstrates that the article is referring to only one object. By way of example, “an element” refers to one element or more than one element.

As used herein, the term “about”, when referring to a value or to an amount of mass, weight, time, volume, concentration, or percentage is meant to encompass variations of in some embodiments ±20% or ±10%, in some embodiments ±5%, in some embodiments ±1%, and in some embodiments ±0.1% from the specified amount, as such variations are appropriate to practice the presently disclosed subject matter. Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the terms “amino acid” and “amino acid residue” are used interchangeably and refer to any of the twenty naturally occurring amino acids, as well as analogs, derivatives, and congeners thereof; amino acid analogs having variant side chains; and all stereoisomers of any of any of the foregoing. Thus, the term “amino acid” is intended to embrace all molecules, whether natural or synthetic, which include both an amino functionality and an acid functionality and capable of being included in a polymer of naturally occurring amino acids.

An amino acid is formed upon chemical digestion (hydrolysis) of a polypeptide at its peptide linkages. The amino acid residues described herein are in one embodiment in the “L” isomeric form. However, residues in the “D” isomeric form can be substituted for any L-amino acid residue, as long as the desired functional property is retained by the polypeptide. NH₂ refers to the free amino group present at the amino terminus of a polypeptide. COOH refers to the free carboxy group present at the carboxy terminus of a polypeptide. In keeping with standard polypeptide nomenclature abbreviations for amino acid residues are shown in tabular form presented hereinabove.

It is noted that all amino acid residue sequences represented herein by formulae have a left-to-right orientation in the conventional direction of amino terminus to carboxy terminus. In addition, the phrases “amino acid” and “amino acid residue” are broadly defined to include modified and unusual amino acids.

Furthermore, it is noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

As used herein, the term “cell” is used in its usual biological sense. In one embodiment, the cell is present in an organism, for example, a plant including, but not limited to poplar, pine, eucalyptus, sweetgum, and other tree species; tobacco; Arabidopsis; rice; corn; wheat; cotton; potato; and cucumber. The cell can be eukaryotic (e.g., a plant cell, such as a tobacco cell or a cell from a tree) or prokaryotic (e.g. a bacterium). The cell can be of somatic or germ line origin, totipotent or pluripotent, dividing or non-dividing. The cell can also be derived from or can comprise a gamete or embryo, a stem cell, or a fully differentiated cell.

As used herein, the terms “host cells” and “recombinant host cells” are used interchangeably and refer cells (for example, plant cells) into which the compositions of the presently disclosed subject matter (for example, an expression vector) can be introduced. Furthermore, the terms refer not only to the particular plant cell into which an expression construct is initially introduced, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the term “gene” refers to a nucleic acid that encodes an RNA, for example, nucleic acid sequences including, but not limited to, structural genes encoding a polypeptide. The target gene can be a gene derived from a cell, an endogenous gene, a transgene, or exogenous genes such as genes of a pathogen, for example a virus, which is present in the cell after infection thereof. The cell containing the target gene can be derived from or contained in any organism, for example a plant, animal, protozoan, virus, bacterium, or fungus. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation from one or more existing sequences.

As is understood in the art, a gene comprises a coding strand and a non-coding strand. As used herein, the terms “coding strand” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

As used herein, the terms “complementarity” and “complementary” refer to a nucleic acid that can form one or more hydrogen bonds with another nucleic acid sequence by either traditional Watson-Crick or other non-traditional types of interactions. In reference to the nucleic molecules of the presently disclosed subject matter, the binding free energy for a nucleic acid molecule with its complementary sequence is sufficient to allow the relevant function of the nucleic acid to proceed, in one embodiment, RNAi activity. For example, the degree of complementarity between the sense and antisense strands of the siRNA construct can be the same or different from the degree of complementarity between the antisense strand of the siRNA and the target nucleic acid sequence. Complementarity to the target sequence of less than 100% in the antisense strand of the siRNA duplex, including point mutations, is not well tolerated when these changes are located between the 3′-end and the middle of the antisense siRNA, whereas mutations near the 5′-end of the antisense siRNA strand can exhibit a small degree of RNAi activity (Elbashir et al., 2001c). Determination of binding free energies for nucleic acid molecules is well known in the art. See e.g., Freier et al., 1986; Turner et al., 1987.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The terms “100% complementary”, “fully complementary”, and “perfectly complementary” indicate that all of the contiguous residues of a nucleic acid sequence can hydrogen bond with the same number of contiguous residues in a second nucleic acid sequence.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA synthesis, processing, and transport, as well as polypeptide synthesis, transport, and post-translational modification of polypeptides. Additionally, processes that affect protein-protein interactions within the cell can also affect gene expression as defined herein.

As used herein, the term “isolated” refers to a molecule substantially free of other nucleic acids, proteins, lipids, carbohydrates, and/or other materials with which it is normally associated, such association being either in cellular material or in a synthesis medium. Thus, the term “isolated nucleic acid” refers to a polynucleotide of genomic, cDNA, or synthetic origin or some combination thereof, which (1) is not associated with the cell in which the “isolated nucleic acid” is found in nature, or (2) is operatively linked to a polynucleotide to which it is not linked in nature. Similarly, the term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated”, when used in the context of an “isolated cell”, refers to a cell that has been removed from its natural environment, for example, as a part of an organ, tissue, or organism.

As used herein, the terms “label” and “labeled” refer to the attachment of a moiety, capable of detection by spectroscopic, radiologic, or other methods, to a probe molecule. Thus, the terms “label” or “labeled” refer to incorporation or attachment, optionally covalently or non-covalently, of a detectable marker into a molecule, such as a polypeptide. Various methods of labeling polypeptides are known in the art and can be used. Examples of labels for polypeptides include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for antibodies, metal binding domains, epitope tags). In some embodiments, labels are attached by spacer arms of various lengths to reduce potential steric hindrance.

As used herein, the term “modulate” refers to an increase, decrease, or other alteration of any, or all, chemical and biological activities or properties of a biochemical entity, e.g., a wild-type or mutant nucleic acid molecule. For example, the term “modulate” can refer to a change in the expression level of a gene or a level of an RNA molecule or equivalent RNA molecules encoding one or more proteins or protein subunits; or to an activity of one or more proteins or protein subunits that is up regulated or down regulated, such that expression, level, or activity is greater than or less than that observed in the absence of the modulator. For example, the term “modulate” can mean “inhibit” or “suppress”, but the use of the word “modulate” is not limited to this definition.

As used herein, the terms “inhibit”, “suppress”, “down regulate”, and grammatical variants thereof are used interchangeably and refer to an activity whereby gene expression or a level of an RNA encoding one or more gene products is reduced below that observed in the absence of a nucleic acid molecule of the presently disclosed subject matter. In one embodiment, inhibition with an siRNA molecule results in a decrease in the steady state level of a target RNA. In another embodiment, inhibition with a siRNA molecule results in an expression level of a target gene that is below that level observed in the presence of an inactive or attenuated molecule that is unable to mediate an RNAi response. In another embodiment, inhibition of gene expression with an siRNA molecule of the presently disclosed subject matter is greater in the presence of the siRNA molecule than in its absence. In still another embodiment, inhibition of gene expression is associated with an enhanced rate of degradation of the mRNA encoded by the gene (for example, by RNAi mediated by an siRNA).

The term “modulation” as used herein refers to both upregulation (i.e., activation or stimulation) and downregulation (i.e., inhibition or suppression) of a response. Thus, the term “modulation”, when used in reference to a functional property or biological activity or process (e.g., enzyme activity or receptor binding), refers to the capacity to upregulate (e.g., activate or stimulate), downregulate (e.g., inhibit or suppress), or otherwise change a quality of such property, activity, or process. In certain instances, such regulation can be contingent on the occurrence of a specific event, such as activation of a signal transduction pathway, and/or can be manifest only in particular cell types.

The term “modulator” refers to a polypeptide, nucleic acid, macromolecule, complex, molecule, small molecule, compound, species, or the like (naturally occurring or non-naturally occurring), or an extract made from biological materials such as bacteria, plants, fungi, or animal cells or tissues, that can be capable of causing modulation. Modulators can be evaluated for potential activity as inhibitors or activators (directly or indirectly) of a functional property, biological activity or process, or a combination thereof (e.g., agonist, partial antagonist, partial agonist, inverse agonist, antagonist, anti-microbial agents, inhibitors of microbial infection or proliferation, and the like), by inclusion in assays. In such assays, many modulators can be screened at one time. The activity of a modulator can be known, unknown, or partially known.

Modulators can be either selective or non-selective. As used herein, the term “selective” when used in the context of a modulator (e.g. an inhibitor) refers to a measurable or otherwise biologically relevant difference in the way the modulator interacts with one molecule (e.g. a gene of interest) versus another similar but not identical molecule (e.g. a member of the same gene family as the gene of interest).

It must be understood that it is not required that the degree to which the interactions differ be completely opposite. Put another way, the term selective modulator encompasses not only those molecules that only bind to mRNA transcripts from a gene of interest and not those of related family members. The term is also intended to include modulators that are characterized by interactions with transcripts from genes of interest and from related family members that differ to a lesser degree. For example, selective modulators include modulators for which conditions can be found (such as the degree of sequence identity) that would allow a biologically relevant difference in the binding of the modulator to transcripts form the gene of interest versus transcripts from related genes.

When a selective modulator is identified, the modulator will bind to one molecule (for example an mRNA transcript of a gene of interest) in a manner that is different (for example, stronger) than it binds to another molecule (for example, an mRNA transcript of a gene related to the gene of interest). As used herein, the modulator is said to display “selective binding” or “preferential binding” to the molecule to which it binds more strongly.

As used herein, the term “mutation” carries its traditional connotation and refers to a change, inherited, naturally occurring or introduced, in a nucleic acid or polypeptide sequence, and is used in its sense as generally known to those of skill in the art.

The term “naturally occurring”, as applied to an object, refers to the fact that an object can be found in nature. For example, a polypeptide or polynucleotide sequence that is present in an organism (including bacteria) that can be isolated from a source in nature and which has not been intentionally modified by man in the laboratory is naturally occurring.

As used herein, the terms “nucleic acid” and “nucleic acid molecule” refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally occurring nucleotides (such as deoxyribonucleotides and ribonucleotides), or analogs of naturally occurring nucleotides (e.g., α-enantiomeric forms of naturally occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes so-called “peptide nucleic acids”, which comprise naturally occurring or modified nucleic acid bases attached to a polyamide backbone. Nucleic acids can be either single stranded or double stranded.

The term “operatively linked”, when describing the relationship between two nucleic acid regions, refers to a juxtaposition wherein the regions are in a relationship permitting them to function in their intended manner. For example, a control sequence “operatively linked” to a coding sequence is ligated in such a way that expression of the coding sequence is achieved under conditions compatible with the control sequences, such as when the appropriate molecules (e.g., inducers and polymerases) are bound to the control or regulatory sequence(s). Thus, in one embodiment, the phrase “operatively linked” refers to a promoter connected to a coding sequence in such a way that the transcription of that coding sequence is controlled and regulated by that promoter. Techniques for operatively linking a promoter to a coding sequence are well known in the art; the precise orientation and location relative to a coding sequence of interest is dependent, inter alia, upon the specific nature of the promoter.

Thus, the term “operatively linked” can refer to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operatively linked. Techniques for operatively linking a promoter region to a nucleotide sequence are known in the art.

The term “operatively linked” can also refer to a transcription termination sequence that is connected to a nucleotide sequence in such a way that termination of transcription of that nucleotide sequence is controlled by that transcription termination sequence. In one embodiment, a transcription termination sequence comprises a sequence that causes transcription by an RNA polymerase III to terminate at the third or fourth T in the terminator sequence, TTTTTTT, therefore the nascent small transcript has 3 or 4 U's at the 3′ terminus.

The phrases “percent identity” and “percent identical,” in the context of two nucleic acid or protein sequences, refer to two or more sequences or subsequences that have in one embodiment at least 60%, in another embodiment at least 70%, in another embodiment at least 80%, in another embodiment at least 85%, in another embodiment at least 90%, in another embodiment at least 95%, in another embodiment at least 98%, and in yet another embodiment at least 99% nucleotide or amino acid residue identity, when compared and aligned for maximum correspondence, as measured using one of the following sequence comparison algorithms or by visual inspection. The percent identity exists in one embodiment over a region of the sequences that is at least about 50 residues in length, in another embodiment over a region of at least about 100 residues, and in still another embodiment the percent identity exists over at least about 150 residues. In yet another embodiment, the percent identity exists over the entire length of a given region, such as a coding region.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm described in Smith & Waterman 1981, by the homology alignment algorithm described in Needleman & Wunsch 1970, by the search for similarity method described in Pearson & Lipman 1988, by computerized implementations of these algorithms (GAP, BESTFIT, FASTA, and TFASTA in the GCG Wisconsin Package, available from Accelrys, Inc., San Diego, Calif., United States of America), or by visual inspection. See generally, Ausubel et al., 1989.

One example of an algorithm that is suitable for determining percent sequence identity and sequence similarity is the BLAST algorithm, which is described in Altschul et al., 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in one embodiment less than about 0.1, in another embodiment less than about 0.01, and in still another embodiment less than about 0.001.

The term “substantially identical”, in the context of two nucleotide sequences, refers to two or more sequences or subsequences that have in one embodiment at least about 80% nucleotide identity, in another embodiment at least about 85% nucleotide identity, in another embodiment at least about 90% nucleotide identity, in another embodiment at least about 95% nucleotide identity, in another embodiment at least about 98% nucleotide identity, and in yet another embodiment at least about 99% nucleotide identity, when compared and aligned for maximum correspondence, as measured using one of the following sequence comparison algorithms or by visual inspection. In some embodiments, the substantial identity exists in nucleotide sequences of at least 50 residues, in some embodiments in nucleotide sequence of at least about 100 residues, in some embodiments in nucleotide sequences of at least about 150 residues, and in some embodiments in nucleotide sequences comprising complete coding sequences. In one aspect, polymorphic sequences can be substantially identical sequences. The term “polymorphic” refers to the occurrence of two or more genetically determined alternative sequences or alleles in a population. An allelic difference can be as small as one base pair. Nonetheless, one of ordinary skill in the art would recognize that the polymorphic sequences correspond to the same gene.

Another indication that two nucleotide sequences are substantially identical is that the two molecules specifically or substantially hybridize to each other under stringent conditions. In the context of nucleic acid hybridization, two nucleic acid sequences being compared can be designated a “probe sequence” and a “target sequence”. A “probe sequence” is a reference nucleic acid molecule, and a ““target sequence” is a test nucleic acid molecule, often found within a heterogeneous population of nucleic acid molecules. A “target sequence” is synonymous with a “test sequence”.

An exemplary nucleotide sequence employed for hybridization studies or assays includes probe sequences that are complementary to or mimic in one embodiment at least an about 14 to 40 nucleotide sequence of a nucleic acid molecule of the presently disclosed subject matter. In some embodiments, probes comprise 14 to 20 nucleotides, or even longer where desired, such as 30, 40, 50, 60, 100, 200, 300, or 500 nucleotides or up to the full length of a given gene. Such fragments can be readily prepared by, for example, directly synthesizing the fragment by chemical synthesis, by application of nucleic acid amplification technology, or by introducing selected sequences into recombinant vectors for recombinant production.

The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

Hybridization can be carried out in 5×SSC, 4×SSC, 3×SSC, 2×SSC, 1×SSC, or 0.2×SSC for at least about 1 hour, 2 hours, 5 hours, 12 hours, or 24 hours (see Sambrook & Russell, 2001, for a description of SSC buffer and other stringency conditions). The temperature of the hybridization can be increased to adjust the stringency of the reaction, for example, from about 25° C. (room temperature), to about 45° C., 50° C., 55° C., 60° C., or 65° C. The hybridization reaction can also include another agent affecting the stringency, for example, hybridization conducted in the presence of 50% formamide increases the stringency of hybridization at a defined temperature.

The hybridization reaction can be followed by a single wash step, or two or more wash steps, which can be at the same or a different salinity and temperature. For example, the temperature of the wash can be increased to adjust the stringency from about 25° C. (room temperature), to about 45° C., 50° C., 55° C., 60° C., 65° C., or higher. The wash step can be conducted in the presence of a detergent, e.g., 0.1 or 0.2% SDS. For example, hybridization can be followed by two wash steps at 65° C. each for about 20 minutes in 2×SSC, 0.1% SDS, and optionally two additional wash steps at 65° C. each for about 20 minutes in 0.2×SSC, 0.1% SDS.

The following are examples of hybridization and wash conditions that can be used to clone homologous nucleotide sequences that are substantially identical to reference nucleotide sequences of the presently disclosed subject matter: a probe nucleotide sequence hybridizes in some embodiments to a target nucleotide sequence in 7% sodium dodecyl sulfate (SDS), 0.5M NaPO₄, 1 mm EDTA at 50° C. followed by washing in 2×SSC, 0.1% SDS at 50° C.; in some embodiments, a probe and target sequence hybridize in 7% sodium dodecyl sulfate (SDS), 0.5M NaPO₄, 1 mm EDTA at 50° C. followed by washing in 1×SSC, 0.1% SDS at 50° C.; in some embodiments, a probe and target sequence hybridize in 7% sodium dodecyl sulfate (SDS), 0.5M NaPO₄, 1 mm EDTA at 50° C. followed by washing in 0.5×SSC, 0.1% SDS at 50° C.; in some embodiments, a probe and target sequence hybridize in 7% sodium dodecyl sulfate (SDS), 0.5M NaPO₄, 1 mm EDTA at 50° C. followed by washing in 0.1×SSC, 0.1% SDS at 50° C.; and in some embodiments, a probe and target sequence hybridize in 7% sodium dodecyl sulfate (SDS), 0.5M NaPO₄, 1 mm EDTA at 50° C. followed by washing in 0.1×SSC, 0.1% SDS at 65° C.

Additional exemplary stringent hybridization conditions include overnight hybridization at 42° C. in a solution comprising, or consisting of, 50% formamide, 10× Denhardt's (0.2% Ficoll, 0.2% polyvinylpyrrolidone, 0.2% bovine serum albumin) and 200 mg/ml of denatured carrier DNA, e.g., sheared salmon sperm DNA, followed by two wash steps at 65° C. each for about 20 minutes in 2×SSC, 0.1% SDS, and two wash steps at 65° C. each for about 20 minutes in 0.2×SSC, 0.1% SDS.

Hybridization can include hybridizing two nucleic acids in solution, or a nucleic acid in solution to a nucleic acid attached to a solid support, e.g., a filter. When one nucleic acid is on a solid support, a prehybridization step can be conducted prior to hybridization. Prehybridization can be carried out for at least about 1 hour, 3 hours, or 10 hours in the same solution and at the same temperature as the hybridization (but without the complementary polynucleotide strand).

Appropriate stringency conditions are known to those skilled in the art or can be determined experimentally by the skilled artisan. See e.g., Ausubel et al., 1989; Sambrook & Russell, 2001; Agrawal, 1993; Tijssen, 1993; Tibanyenda et al., 1984; and Ebel et al., 1992.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

The term “phenotype” refers to the entire physical, biochemical, and physiological makeup of a cell or an organism, e.g., having any one trait or any group of traits. As such, phenotypes result from the expression of genes within a cell or an organism, and relate to traits that are potentially observable or assayable.

As used herein, the terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product. The term “polypeptide” encompasses proteins of all functions, including enzymes. Thus, exemplary polypeptides include gene products, naturally occurring proteins, homologs, orthologs, paralogs, fragments, and other equivalents, variants and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8 or 10 amino acids long, at least 14 amino acids long, at least 20, 30, 40 or 50 amino acids long, at least 75 amino acids long, or at least 100, 150, 200, 300, 500 or more amino acids long. A fragment can retain one or more of the biological activities of the reference polypeptide. Further, fragments can include a sub-fragment of a specific region, which sub-fragment retains a function of the region from which it is derived.

As used herein, the term “primer” refers to a sequence comprising in one embodiment two or more deoxyribonucleotides or ribonucleotides, in another embodiment more than three, in another embodiment more than eight, and in yet another embodiment at least about 20 nucleotides of an exonic or intronic region. Such oligonucleotides are in one embodiment between ten and thirty bases in length.

The term “purified” refers to an object species that is the predominant species present (i.e., on a molar basis it is more abundant than any other individual species in the composition). A “purified fraction” is a composition wherein the object species comprises at least about 50 percent (on a molar basis) of all species present. In making the determination of the purity of a species in solution or dispersion, the solvent or matrix in which the species is dissolved or dispersed is usually not included in such determination; instead, only the species (including the one of interest) dissolved or dispersed are taken into account. Generally, a purified composition will have one species that comprises more than about 80 percent of all species present in the composition, more than about 85%, 90%, 95%, 99% or more of all species present. The object species can be purified to essential homogeneity (contaminant species cannot be detected in the composition by conventional detection methods) wherein the composition consists essentially of a single species. A skilled artisan can purify a polypeptide of the presently disclosed subject matter using standard techniques for protein purification in light of the teachings herein. Purity of a polypeptide can be determined by a number of methods known to those of skill in the art, including for example, amino-terminal amino acid sequence analysis, gel electrophoresis, and mass-spectrometry analysis.

A “reference sequence” is a defined sequence used as a basis for a sequence comparison. A reference sequence can be a subset of a larger sequence, for example, as a segment of a full-length nucleotide or amino acid sequence, or can comprise a complete sequence. Generally, when used to refer to a nucleotide sequence, a reference sequence is at least 200, 300 or 400 nucleotides in length, frequently at least 600 nucleotides in length, and often at least 800 nucleotides in length. Because two proteins can each (1) comprise a sequence (i.e., a portion of the complete protein sequence) that is similar between the two proteins, and (2) can further comprise a sequence that is divergent between the two proteins, sequence comparisons between two (or more) proteins are typically performed by comparing sequences of the two proteins over a “comparison window” (defined hereinabove) to identify and compare local regions of sequence similarity.

The term “regulatory sequence” is a generic term used throughout the specification to refer to polynucleotide sequences, such as initiation signals, enhancers, regulators, promoters, and termination sequences, which are necessary or desirable to affect the expression of coding and non-coding sequences to which they are operatively linked. Exemplary regulatory sequences are described in Goeddel, 1990, and include, for example, the early and late promoters of simian virus 40 (SV40), adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. The nature and use of such control sequences can differ depending upon the host organism. In prokaryotes, such regulatory sequences generally include promoter, ribosomal binding site, and transcription termination sequences. The term “regulatory sequence” is intended to include, at a minimum, components the presence of which can influence expression, and can also include additional components the presence of which is advantageous, for example, leader sequences and fusion partner sequences.

In certain embodiments, transcription of a polynucleotide sequence is under the control of a promoter sequence (or other regulatory sequence) that controls the expression of the polynucleotide in a cell-type in which expression is intended. It will also be understood that the polynucleotide can be under the control of regulatory sequences that are the same or different from those sequences which control expression of the naturally occurring form of the polynucleotide. In one embodiment, a promoter sequence is a DNA-dependent RNA polymerase III promoter (e.g. a promoter for an H1, 5S, or U6 gene, or an Arabidopsis thaliana At7SL4 gene promoter, such as that disclosed as SEQ ID NO: 3). In another embodiment, a promoter sequence is selected from the group consisting of an adenovirus VA1 promoter sequence, a Vault promoter sequence, a telomerase RNA promoter sequence, and a tRNA gene promoter sequence. It is understood that the entire promoter identified for any promoter (for example, the promoters listed herein) need not be employed, and that a functional derivative thereof can be used. As used herein, the phrase “functional derivative” refers to a nucleic acid sequence that comprises sufficient sequence to direct transcription of another operatively linked nucleic acid molecule. As such, a “functional derivative” can function as a minimal promoter, as that term is defined herein.

Termination of transcription of a polynucleotide sequence is typically regulated by an operatively linked transcription termination sequence (for example, an RNA polymerase III termination sequence). In certain instances, transcriptional terminators are also responsible for correct mRNA polyadenylation. The 3′ non-transcribed regulatory DNA sequence includes from in one embodiment about 50 to about 1,000, and in another embodiment about 100 to about 1,000, nucleotide base pairs and contains plant transcriptional and translational termination sequences. Appropriate transcriptional terminators and those that are known to function in plants include the CaMV ³⁵S terminator, the tml terminator, the nopaline synthase terminator, the pea rbcS E9 terminator, the terminator for the T7 transcript from the octopine synthase gene of Agrobacterium tumefaciens, and the 3′ end of the protease inhibitor I or II genes from potato or tomato, although other 3′ elements known to those of skill in the art can also be employed. Alternatively, a gamma coixin, oleosin 3, or other terminator from the genus Coix can be used. In one embodiment, an RNA polymerase III termination sequence comprises the nucleotide sequence TTTTTTT.

The term “reporter gene” refers to a nucleic acid comprising a nucleotide sequence encoding a protein that is readily detectable either by its presence or activity, including, but not limited to, luciferase, fluorescent protein (e.g., green fluorescent protein), chloramphenicol acetyl transferase, β-galactosidase, secreted placental alkaline phosphatase, β-lactamase, human growth hormone, and other secreted enzyme reporters. Generally, a reporter gene encodes a polypeptide not otherwise produced by the host cell, which is detectable by analysis of the cell(s), e.g., by the direct fluorometric, radioisotopic or spectrophotometric analysis of the cell(s) and typically without the need to kill the cells for signal analysis. In certain instances, a reporter gene encodes an enzyme, which produces a change in fluorometric properties of the host cell, which is detectable by qualitative, quantitative, or semiquantitative function or transcriptional activation. Exemplary enzymes include esterases, β-lactamase, phosphatases, peroxidases, proteases (tissue plasminogen activator or urokinase) and other enzymes whose function can be detected by appropriate chromogenic or fluorogenic substrates known to those skilled in the art or developed in the future.

As used herein, the term “sequencing” refers to determining the ordered linear sequence of nucleic acids or amino acids of a DNA or protein target sample, using conventional manual or automated laboratory techniques.

As used herein, the term “substantially pure” refers to that the polynucleotide or polypeptide is substantially free of the sequences and molecules with which it is associated in its natural state, and those molecules used in the isolation procedure. The term “substantially free” refers to that the sample is in one embodiment at least 50%, in another embodiment at least 70%, in another embodiment 80% and in still another embodiment 90% free of the materials and compounds with which is it associated in nature.

As used herein, the term target cell” refers to a cell, into which it is desired to insert a nucleic acid sequence or polypeptide, or to otherwise effect a modification from conditions known to be standard in the unmodified cell. A nucleic acid sequence introduced into a target cell can be of variable length. Additionally, a nucleic acid sequence can enter a target cell as a component of a plasmid or other vector or as a naked sequence.

As used herein, the term “target gene” refers to a gene expressed in a cell the expression of which is targeted for modulation using the methods and compositions of the presently disclosed subject matter. A target gene, therefore, comprises a nucleic acid sequence corresponding to the sequence of an siRNA. Similarly, the terms “target RNA” or “target mRNA” refers to the transcript of a target gene to which the siRNA is intended to bind, leading to modulation of the expression of the target gene.

As used herein, the term “transcription” refers to a cellular process involving the interaction of an RNA polymerase with a gene that directs the expression as RNA of the structural information present in the coding sequences of the gene. The process includes, but is not limited to, the following steps: (a) the transcription initiation; (b) transcript elongation; (c) transcript splicing; (d) transcript capping; (e) transcript termination; (f) transcript polyadenylation; (g) nuclear export of the transcript; (h) transcript editing; and (i) stabilizing the transcript.

As used herein, the term “transcription factor” refers to a cytoplasmic or nuclear protein which binds to a gene, or binds to an RNA transcript of a gene, or binds to another protein which binds to a gene or an RNA transcript or another protein which in turn binds to a gene or an RNA transcript, so as to thereby modulate expression of the gene. Such modulation can additionally be achieved by other mechanisms; the essence of a “transcription factor for a gene” pertains to a factor that alters the level of transcription of the gene in some way.

The term “transfection” refers to the introduction of a nucleic acid, e.g., an expression vector, into a recipient cell, which in certain instances involves nucleic acid-mediated gene transfer. The term “transformation” refers to a process in which a cell's genotype is changed as a result of the cellular uptake of exogenous nucleic acid. For example, a transformed cell can express a recombinant form of a polypeptide of the presently disclosed subject matter.

The transformation of a cell with an exogenous nucleic acid (for example, an expression vector) can be characterized as transient or stable. As used herein, the term “stable” refers to a state of persistence that is of a longer duration than that which would be understood in the art as “transient”. These terms can be used both in the context of the transformation of cells (for example, a stable transformation), or for the expression of a transgene (for example, the stable expression of a vector-encoded siRNA) in a transgenic cell. In one aspect, a stable transformation results in the incorporation of the exogenous nucleic acid molecule (for example, an expression vector) into the genome of the transformed cell. As a result, when the cell divides, the vector DNA is replicated along with plant genome so that progeny cells also contain the exogenous DNA in their genomes.

In another aspect, the term “stable expression” relates to expression of a nucleic acid molecule (for example, a vector-encoded siRNA) over time. Thus, stable expression requires that the cell into which the exogenous DNA is introduced expresses the encoded nucleic acid at a consistent level over time. Additionally, stable expression can occur over the course of generations. When the expressing cell divides, at least a fraction of the resulting daughter cells can also express the encoded nucleic acid, and at about the same level. It should be understood that it is not necessary that every cell derived from the cell into which the vector was originally introduced express the nucleic acid molecule of interest. Rather, particularly in the context of a whole plant, the term “stable expression” requires only that the nucleic acid molecule of interest be stably expressed in tissue(s) and/or location(s) of the plant in which expression is desired. In one embodiment, stable expression of an exogenous nucleic acid is achieved by the integration of the nucleic acid into the genome of the host cell.

The term “vector” refers to a nucleic acid capable of transporting another nucleic acid to which it has been linked. One type of vector that can be used in accord with the presently disclosed subject matter is an Agrobacterium binary vector, i.e., a nucleic acid capable of integrating the nucleic acid sequence of interest into the host cell (for example, a plant cell) genome. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of plasmids. In the present specification, “plasmid” and “vector” are used interchangeably as the plasmid is the most commonly used form of vector. However, the presently disclosed subject matter is intended to include such other forms of expression vectors which serve equivalent functions and which become known in the art subsequently hereto.

The term “expression vector” as used herein refers to a DNA sequence capable of directing expression of a particular nucleotide sequence in an appropriate host cell, comprising a promoter operatively linked to the nucleotide sequence of interest which is operatively linked to transcription termination sequences. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression. The nucleotide sequence of interest, including any additional sequences designed to effect proper expression of the nucleotide sequences, can also be referred to as an “expression cassette”.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

The term “promoter” or “promoter region” each refers to a nucleotide sequence within a gene that is positioned 5′ to a coding sequence and functions to direct transcription of the coding sequence. The promoter region comprises a transcriptional start site, and can additionally include one or more transcriptional regulatory elements. In one embodiment, a method of the presently disclosed subject matter employs a RNA polymerase III promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the CMV minimal promoter, the HSV-tk minimal promoter, the simian virus 40 (SV40) minimal promoter, the human b-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operatively linked gene. For example, cell-type-specific or tissue-specific transcriptional regulatory elements can be added to minimal promoters to create recombinant promoters that direct transcription of an operatively linked nucleotide sequence in a cell-type-specific or tissue-specific manner. As used herein, the term “minimal promoter” also encompasses a functional derivative of a promoter disclosed herein, including, but not limited to an RNA polymerase III promoter (for example, an H1, 7SL, 5S, or U6 promoter), an adenovirus VA1 promoter, a Vault promoter, a telomerase RNA promoter, and a tRNA gene promoter.

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types. Exemplary constitutive promoters include the promoters for the following genes which encode certain constitutive or “housekeeping” functions: hypoxanthine phosphoribosyl transferase (HPRT), dihydrofolate reductase (DHFR; (Scharfmann et al., 1991), adenosine deaminase, phosphoglycerate kinase (PGK), pyruvate kinase, phosphoglycerate mutase, the p-actin promoter (see e.g., Williams et al., 1993), and other constitutive promoters known to those of skill in the art. “Tissue-specific” or “cell-type-specific” promoters, on the other hand, direct transcription in some tissues and cell types but are inactive in others. Exemplary tissue-specific promoters include those promoters described in more detail hereinbelow, as well as other tissue-specific and cell-type specific promoters known to those of skill in the art.

When used in the context of a promoter, the term “linked” as used herein refers to a physical proximity of promoter elements such that they function together to direct transcription of an operatively linked nucleotide sequence.

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element. In one embodiment, a transcriptional regulatory sequence is a transcription termination sequence, alternatively referred to herein as a transcription termination signal.

The term “transcription factor” generally refers to a protein that modulates gene expression by interaction with the transcriptional regulatory element and cellular components for transcription, including RNA Polymerase, Transcription Associated Factors (TAFs), chromatin-remodeling proteins, and any other relevant protein that impacts gene transcription.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “p-value”. Those p-values that fall below a user-defined cutoff point are regarded as significant. In some embodiments, a p-value less than or equal to 0.05, in some embodiments less than 0.01, in some embodiments less than 0.005, and in some embodiments less than 0.001, are regarded as significant.

As used herein, the phrase “target RNA” refers to an RNA molecule (for example, an mRNA molecule encoding a plant gene product) that is a target for downregulation. Similarly, the phrase “target site” refers to a sequence within a target RNA that is “targeted” for cleavage mediated by an siRNA construct that contains sequences within its antisense strand that are complementary to the target site. Also similarly, the phrase “target cell” refers to a cell that expresses a target RNA and into which an siRNA is intended to be introduced. A target cell is in one embodiment a cell in a plant. For example, a target cell can comprise a target RNA expressed in a plant.

As used herein, the phrase “detectable level of cleavage” refers to a degree of cleavage of target RNA (and formation of cleaved product RNAs) that is sufficient to allow detection of cleavage products above the background of RNAs produced by random degradation of the target RNA. Production of siRNA-mediated cleavage products from at least 1-5% of the target RNA is sufficient to allow detection above background for most detection methods.

The terms “small interfering RNA”, “short interfering RNA”, and “siRNA” are used interchangeably and refer to any nucleic acid molecule capable of mediating RNA interference (RNAi) or post-transcriptional gene silencing. See e.g., Bass, 2001; Elbashir et al., 2001a; and PCT International Publication Nos. WO 00/44895, WO 01/36646, WO 99/32619, WO 00/01846, WO 01/29058, WO 99/07409, and WO 00/44914. In one embodiment, the siRNA comprises a single stranded polynucleotide having self-complementary sense and antisense regions, wherein the antisense region comprises a sequence complementary to a region of a target nucleic acid molecule. In another embodiment, the siRNA comprises a single stranded polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi.

The methods of the presently disclosed subject matter can employ siRNA molecules of the following general structure:

[image]

wherein N is any nucleotide, provided that in the loop structure identified as N₅₋₉ above, all 5-9 nucleotides remain in a single-stranded conformation. Similarly, N₁₋₈ can be any sequence of 1-8 nucleotides or modified nucleotides, provided that the nucleotides remain in a single-stranded conformation in the siRNA molecule. The duplex represented above as “19-30 bases of a plant gene” can be formed using any contiguous 19-30 base sequence of a transcription product of a plant gene. In constructing an siRNA molecule of the presently disclosed subject matter, this 19-30 base sequence is followed (in a 5′ to 3′ direction) by 5-9 random nucleotides (N₅₋₉ above), the reverse-complement of the 19-30 base sequence, and finally 1-8 random nucleotides (N₁₋₈ above).

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of the siRNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs. In one embodiment, the double stranded region is at least 15 basepairs, in another embodiment between 15 and 50 basepairs, and in yet another embodiment between 15 and 30 basepairs. In still another embodiment, the length of the double stranded region is selected from the group consisting of 19, 21, 22, 25, and 30 basepairs. In a representative embodiment, the length of the double stranded region is 19 basepairs. As describe hereinabove, the formation of the double stranded region results from the hybridization of complementary RNA strands (for example, a sense strand and an antisense strand), either via an intermolecular hybridization (i.e., involving 2 or more distinct RNA molecules) or via an intramolecular hybridization, the latter of which can occur when a single RNA molecule contains self-complementary regions that are capable of hybridizing to each other on the same RNA molecule. These self-complementary regions are typically separated by a short stretch of nucleotides (for example, about 5-10 nucleotides) such that the intramolecular hybridization event forms what is referred to in the art as a “hairpin”.

III. Target Genes

The presently disclosed subject matter provides methods for stably modulating expression of plant genes using siRNAs. The methods are applicable to any gene expressed in the plant. In one embodiment, the methods are used to modulate the expression of genes in trees.

In one embodiment, genes associated with lignin biosynthesis are targeted for modulation. Lignin is a major component of wood, and the regulation of its biosynthesis has can have a major impact on paper and pulping processes. Several genes have been identified that are involved in the biosynthesis of lignin including, but not limited to sinapyl alcohol dehydrogenase (SAD), cinnamyl alcohol dehydrogenase (CAD), 4-coumarate:CoA ligase (4CL), cinnamoyl CoA O-methyltransferase (CCoAOMT; also referred to as CCOMT), caffeate O-methyltransferase (COMT), ferulate-5-hydroxylase (F5H), cinnamate-4-hydroxylase (C4H), p-coumarate-3-hydroxylase (C3H), cinnamoyl CoA reductase (CCR), and phenylalanine ammonia lyase (PAL). Reviewed in Anterola & Lewis, 2002; Boerjan et al., 2003. Reduction in the activities of one or more of these genes has been shown to result in reduced lignin deposition (see Anterola & Lewis, 2002; Boerjan et al., 2003), and thus these genes provide potential targets for siRNA-mediated gene expression modulation.

In another embodiment, genes associated with cellulose biosyntheses are targeted for modulation. Representative, non-limiting genes that have been identified that are associated with cellulose biosynthesis include cellulose synthase (CeS; also referred to as CESA in some plants), cellulose synthase-like (CSL), glucosidase, glucan synthase, Korrigan endocellulase, callose synthase, and sucrose synthase.

In other embodiments, other plant genes are targeted for modulation using siRNAs. A non-limiting list of gene families that can be targeted include hormone-related genes including, but not limited to isopentyl transferase (ipt), gibberellic acid (GA) oxidase, auxin (AUX), auxin-responsive and auxin-induced genes, and members of the ROL gene family; disease-related genes, stress-related genes, and transcription factors.

It is understood that the target genes listed hereinabove are exemplary only, and that the methods and compositions of the presently disclosed subject matter can be applied to modulate the expression of any gene in any plant.

IV. Nucleic Acids

The nucleic acid molecules employed in accordance with the presently disclosed subject matter include any nucleic acid molecule encoding a plant gene product, as well as the nucleic acid molecules that are used in accordance with the presently disclosed subject matter to a modulation of the expression of a plant gene. Thus, the nucleic acid molecules employed in accordance with the presently disclosed subject matter include, but are not limited to, the nucleic acid molecules described herein; sequences substantially identical to those described herein; and subsequences and elongated sequences thereof. The presently disclosed subject matter also encompasses genes, cDNAs, chimeric genes, and vectors comprising the disclosed nucleic acid sequences.

An exemplary nucleotide sequence employed in the methods disclosed herein comprises sequences that are complementary to each other, the complementary regions being capable of forming a duplex of, in one embodiment, at least about 15 to 50 basepairs. One strand of the duplex comprises a nucleic acid sequence of at least 15 contiguous bases having a nucleic acid sequence of a nucleic acid molecule of the presently disclosed subject matter. In some embodiments, one strand of the duplex comprises a nucleic acid sequence comprising 15 to 18 nucleotides, or even longer where desired, such as 19, 20, 21, 22, 25, or 30 nucleotides or up to the full length of any of those described herein. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

The term “subsequence” refers to a sequence of a nucleic acid molecule that comprises a part of a longer nucleic acid sequence. An exemplary subsequence is a sequence that comprises part of a duplexed region of an siRNA, one strand of which is complementary to the sequence of an mRNA.

The term “elongated sequence” refers to an addition of nucleotides (or other analogous molecules) incorporated into the nucleic acid. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

Nucleic acids of the presently disclosed subject matter can be cloned, synthesized, recombinantly altered, mutagenized, or subjected to combinations of these techniques. Standard recombinant DNA and molecular cloning techniques used to isolate nucleic acids are known in the art. Exemplary, non-limiting methods are described by Silhavy et al., 1984; Ausubel et al., 1989; Glover & Hames, 1995; and Sambrook & Russell, 2001). Site-specific mutagenesis to create base pair changes, deletions, or small insertions is also known in the art as exemplified by publications (see e.g., Adelman et al., 1983; Sambrook & Russell, 2001).

V. Vectors

In another aspect of the presently disclosed subject matter, siRNA molecules are expressed from transcription units inserted into nucleic acid vectors (alternatively referred to generally as “recombinant vectors” or “expression vectors”). A vector is used to deliver a nucleic acid molecule encoding a short interfering RNA (siRNA) into a plant cell to target a specific plant gene. The recombinant vectors can be, for example, DNA plasmids or viral vectors. Various expression vectors are known in the art. The selection of the appropriate expression vector can be made on the basis of several factors including, but not limited to the cell type wherein expression is desired. For example, Agrobacterium-based expression vectors can be used to express the nucleic acids of the presently disclosed subject matter when stable expression of the vector insert is sought in a plant cell.

V.A. Promoters

The expression of the nucleotide sequence in the expression cassette can be under the control of a constitutive promoter or an inducible promoter that initiates transcription only when the host cell is exposed to some particular external stimulus. For bacterial production of an siRNA, exemplary promoters include Simian virus 40 early promoter, a long terminal repeat promoter from retrovirus, an actin promoter, a heat shock promoter, and a metallothionein protein. For in vivo production of an siRNA in plants, exemplary constitutive promoters are derived from the CaMV ³⁵S, rice actin, and maize ubiquitin genes, each described herein below. Exemplary inducible promoters for this purpose include the chemically inducible PR-1a promoter and a wound-inducible promoter, also described herein below.

Selected promoters can direct expression in specific cell types (such as leaf epidermal cells, mesophyll cells, root cortex cells) or in specific tissues or organs (roots, leaves or flowers, for example). Exemplary tissue-specific promoters include well-characterized root-, pith-, and leaf-specific promoters, each described herein below.

Depending upon the host cell system utilized, any one of a number of suitable promoters can be used. Promoter selection can be based on expression profile and expression level. The following are non-limiting examples of promoters that can be used in the expression cassettes.

V.A.1. Constitutive Expression

³⁵S Promoter. The CaMV ³⁵S promoter can be used to drive constitutive gene expression. Construction of the plasmid pCGN1761 is described in the published patent application EP 0 392 225, which is hereby incorporated by reference. pCGN1761 contains the “double” CaMV ³⁵S promoter and the tml transcriptional terminator with a unique EcoRI site between the promoter and the terminator and has a pUC-type backbone. A derivative of pCGN1761 is constructed which has a modified polylinker that includes NotI and XhoI sites in addition to the existing EcoRI site. This derivative is designated pCGN1761ENX. pCGN1761ENX is useful for the cloning of cDNA sequences or gene sequences (including microbial open reading frame (ORF) sequences) within its polylinker for the purpose of their expression under the control of the ³⁵S promoter in transgenic plants. The entire ³⁵S promoter-gene sequence-tml terminator cassette of such a construction can be excised by HindIII, SphI, SalI, and XbaI sites 5′ to the promoter and XbaI, BamHI and BgII sites 3′ to the terminator for transfer to transformation vectors such as those described below. Furthermore, the double ³⁵S promoter fragment can be removed by 5′ excision with HindIII, SphI, SaII, XbaI, or PstI, and 3′ excision with any of the polylinker restriction sites (EcoRI, NotI or XhoI) for replacement with another promoter.

Actin Promoter. Several isoforms of actin are known to be expressed in most cell types and consequently the actin promoter is a good choice for a constitutive promoter. In particular, the promoter from the rice ActI gene has been cloned and characterized (McElroy et al., 1990). A 1.3 kb fragment of the promoter was found to contain all the regulatory elements required for expression in rice protoplasts. Furthermore, numerous expression vectors based on the ActI promoter have been constructed specifically for use in monocotyledons (McElroy et al., 1991). These incorporate the ActI-intron 1, AdhI 5′ flanking sequence and AdhI-intron 1 (from the maize alcohol dehydrogenase gene) and sequence from the CaMV ³⁵S promoter. Vectors showing highest expression were fusions of ³⁵S and ActI intron or the ActI 5′ flanking sequence and the ActI intron. Optimization of sequences around the initiating ATG (of the GUS reporter gene) also enhanced expression. The promoter expression cassettes described by McElroy et al., 1991 can be easily modified for gene expression and are particularly suitable for use in monocotyledonous hosts. For example, promoter-containing fragments is removed from the McElroy constructions and used to replace the double ³⁵S promoter in pCGN1761ENX, which is then available for the insertion of specific gene sequences. The fusion genes thus constructed can then be transferred to appropriate transformation vectors. In a separate report, the rice ActI promoter with its first intron has also been found to direct high expression in cultured barley cells (Chibbar et al., 1993).

Ubiguitin Promoter. Ubiquitin is another gene product known to accumulate in many cell types and its promoter has been cloned from several species for use in transgenic plants (e.g. sunflower—Binet et al., 1991 and maize—Christensen et al., 1989). The maize ubiquitin promoter has been developed in transgenic monocot systems and its sequence and vectors constructed for monocot transformation are disclosed in the patent publication EP 0 342 926 which is herein incorporated by reference. Taylor et al., 1993 describe a vector (pAHC25) that comprises the maize ubiquitin promoter and first intron and its high activity in cell suspensions of numerous monocotyledons when introduced via microprojectile bombardment. The ubiquitin promoter is suitable for gene expression in transgenic plants, especially monocotyledons. Suitable vectors are derivatives of pAHC25 or any of the transformation vectors described in this application, modified by the introduction of the appropriate ubiquitin promoter and/or intron sequences.

V.A.2. Inducible Expression

Chemically Inducible PR-1a Promoter. The double ³⁵S promoter in pCGN1761ENX can be replaced with any other promoter of choice that will result in suitably high expression levels. By way of example, one of the chemically regulatable promoters described in U.S. Pat. No. 5,614,395 can replace the double 35S promoter. The promoter of choice is preferably excised from its source by restriction enzymes, but can alternatively be PCR-amplified using primers that carry appropriate terminal restriction sites. Should PCR-amplification be undertaken, then the promoter should be re-sequenced to check for amplification errors after the cloning of the amplified promoter in the target vector. The chemical/pathogen regulated tobacco PR-1a promoter is cleaved from plasmid pCIB1004 (for construction, see EP 0 332 104, which is hereby incorporated by reference) and transferred to plasmid pCGN1761ENX (Uknes et al, 1992). pCIB1004 is cleaved with NcoI and the resultant 3′ overhang of the linearized fragment is rendered blunt by treatment with T4 DNA polymerase. The fragment is then cleaved with HindIII and the resultant PR-1a promoter-containing fragment is gel purified and cloned into pCGN1761 ENX from which the double ³⁵S promoter has been removed. This is done by cleavage with XhoI and blunting with T4 DNA polymerase, followed by cleavage with HindIII and isolation of the larger vector-terminator-containing fragment into which the pCIB1004 promoter fragment is cloned. This generates a pCGN1761ENX derivative with the PR-1a promoter and the tml terminator and an intervening polylinker with unique EcoRI and NotI sites. The selected coding sequence can be inserted into this vector, and the fusion products (i.e., promoter-gene-terminator) can subsequently be transferred to any selected transformation vector, including those described below. Various chemical regulators can be employed to induce expression of the selected coding sequence in the plants transformed according to the presently disclosed subject matter, including the benzothiadiazole, isonicotinic acid, and salicylic acid compounds disclosed in U.S. Pat. Nos. 5,523,311 and 5,614,395, herein incorporated by reference.

Wound-inducible Promoters. Wound-inducible promoters can also be suitable for gene expression. Numerous such promoters have been described (e.g. Xu et al., 1993; Logemann et al., 1989; Rohrmeier & Lehle, 1993; Firek et al., 1993; Warner et al., 1993) and all are suitable for use with the presently disclosed subject matter. Logemann et al., 1989 describe the 5′ upstream sequences of the dicotyledonous potato wunI gene. Xu et al., 1993 show that a wound-inducible promoter from the dicotyledon potato (pin2) is active in the monocotyledon rice. Further, Rohrmeier & Lehle, 1993 describe the cloning of the maize Wipl cDNA, which is wound induced and which can be used to isolate the cognate promoter using standard techniques. Similarly, Firek et al., 1993 and Warner et al., 1993 have described a wound-induced gene from the monocotyledon Asparagus officinalis, which is expressed at local wound and pathogen invasion sites. Using cloning techniques well known in the art, these promoters can be transferred to suitable vectors, fused to the genes pertaining to the presently disclosed subject matter, and used to express these genes at the sites of plant wounding.

V.A.3. Tissue-Specific Expression

Root Promoter. Another pattern of gene expression is root expression. A suitable root promoter is described by de Framond, 1991 and also in the published patent application EP 0 452 269, which is herein incorporated by reference. This promoter is transferred to a suitable vector such as pCGN1761 ENX for the insertion of a selected gene and subsequent transfer of the entire promoter-gene-terminator cassette to a transformation vector of interest.

Pith Promoter. PCT International Publication No. WO 93/07278, which is herein incorporated by reference, describes the isolation of the maize trpA gene, which is preferentially expressed in pith cells. The gene sequence and promoter extending up to −1726 bp from the start of transcription are presented. Using standard molecular biological techniques, this promoter, or parts thereof, can be transferred to a vector such as pCGN1761 where it can replace the ³⁵S promoter and be used to drive the expression of a foreign gene in a pith-preferred manner. In fact, fragments containing the pith-preferred promoter or parts thereof can be transferred to any vector and modified for utility in transgenic plants.

Leaf Promoter. A maize gene encoding phosphoenol carboxylase (PEPC) has been described by Hudspeth & Grula, 1989. Using standard molecular biological techniques the promoter for this gene can be used to drive the expression of any gene in a leaf-specific manner in transgenic plants.

V.B. Transcriptional Terminators A variety of transcriptional terminators are available for use in expression cassettes. These are responsible for the termination of transcription beyond the transgene and its correct polyadenylation. Appropriate transcriptional terminators are those that are known to function in plants and include the CaMV ³⁵S terminator, the tml terminator, the nopaline synthase terminator, and the pea rbcS E9 terminator. With regard to RNA polymerase III terminators, these terminators typically comprise a run of 5 or more consecutive thymidine residues. In one embodiment, an RNA polymerase III terminator comprises the sequence TTTTTTT. These can be used in both monocotyledons and dicotyledons.

V.C. Sequences for the Enhancement or Regulation of Expression Numerous sequences have been found to enhance the expression of an operatively lined nucleic acid sequence, and these sequences can be used in conjunction with the nucleic acids of the presently disclosed subject matter to increase their expression in transgenic plants.

Various intron sequences have been shown to enhance expression, particularly in monocotyledonous cells. For example, the introns of the maize AdhI gene have been found to significantly enhance the expression of the wild-type gene under its cognate promoter when introduced into maize cells. Intron 1 was found to be particularly effective and enhanced expression in fusion constructs with the chloramphenicol acetyltransferase gene (Callis et al., 1987). In the same experimental system, the intron from the maize bronze I gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV, the “W-sequence”), Maize Chlorotic Mottle Virus (MCMV), and Alfalfa Mosaic Virus (AMV) have been shown to be effective in enhancing expression (e.g. Gallie et al., 1987; Skuzeski et al., 1990).

VI. Recombinant Expression Vectors

Suitable expression vectors that can be used include, but are not limited to, the following vectors or their derivatives: yeast vectors, bacteriophage vectors (e.g., lambda phage), and plasmid and cosmid DNA vectors.

Numerous vectors available for plant transformation can be prepared and employed in the present methods. Exemplary vectors include pCIB200, pCIB2001, pCIB10, pCIB3064, pSOG19, pSOG35, and pSIT, each described herein. The selection of vector can depend upon the chosen transformation technique and the target species for transformation.

VI.A. Agrobacterium Transformation Vectors

Many vectors are available for transformation using Agrobacterium tumefaciens. These typically carry at least one T-DNA border sequence and include vectors such as pBIN19 (Bevan, 1984) and pXYZ. Below, the construction of two typical vectors suitable for Agrobacterium transformation is described.

PCIB200 and PCIB2001. The binary vectors pcIB200 and pCIB2001 are used for the construction of recombinant vectors for use with Agrobacterium and are constructed in the following manner. pTJS75kan is created by Narl digestion of pTJS75 (Schmidhauser & Helinski, 1985) allowing excision of the tetracycline-resistance gene, followed by insertion of an Accl fragment from pUC4K carrying an NPTII (Messing & Vierra, 1982; Bevan et al., 1983; McBride et al., 1990). XhoI linkers are ligated to the EcoRV fragment of PCIB7 which contains the left and right T-DNA borders, a plant selectable nos/nptlI chimeric gene and the pUC polylinker (Rothstein et al., 1987), and the XhoI-digested fragment are cloned into SalI-digested pTJS75kan to create pCIB200 (see also EP 0 332 104, herein incorporated by reference).

pCIB200 contains the following unique polylinker restriction sites: EcoRI, Sstl, KpnI, BglII, XbaI, and SalI. pCIB2001 is a derivative of pCIB200 created by the insertion into the polylinker of additional restriction sites. Unique restriction sites in the polylinker of pCIB2001 are EcoRI, Sstl, KpnI, BglII, XbaI, SalI, Mlul, Bcll, Avril, ApaI, HpaI, and StuI. pCIB2001, in addition to containing these unique restriction sites also has plant and bacterial kanamycin selection, left and right T-DNA borders for Agrobacterium-mediated transformation, the RK2-derived trfA function for mobilization between E. coli and other hosts, and the OriT and OriV functions also from RK2. The pCIB2001 polylinker is suitable for the cloning of plant expression cassettes containing their own regulatory signals.

pCIB10 and Hygromycin Selection Derivatives thereof. The binary vector pCIB10 contains a gene encoding kanamycin resistance for selection in plants and T-DNA right and left border sequences and incorporates sequences from the wide host-range plasmid pRK252 allowing it to replicate in both E. coli and Agrobacterium. Its construction is described by Rothstein et al., 1987. Various derivatives of pCIB10 are constructed which incorporate the gene for hygromycin B phosphotransferase described by Gritz et al., 1983. These derivatives enable selection of transgenic plant cells on hygromycin only (pCIB743), or hygromycin and kanamycin (pCIB715, pCIB717).

pSIT. pSIT is an Agrobacterium binary vector that can be used to stably express exogenous nucleic acids (for example, siRNAs) in plants. pSIT encodes two transcription units. The first is a transcription unit encoding a selectable marker under control of a promoter-transcription terminator pair that functions in plants cells. The second transcription unit encodes the gene of interest (for example, an siRNA) under the control of a second promoter-transcription terminator pair, which specifically directs the transcription to generate a functional siRNA in plant cells and which can be the same or different than the one operatively linked to the selectable marker. In one embodiment, an siRNA is operatively linked to an RNA polymerase III promoter (for example, the At7SL4 promoter) and the RNA-polymerase-III-recognized transcription terminator (for example, TTTTTTT). The integration of the siRNA cassette is guaranteed if the transformants survived through the antibiotic selection process due to the expression of the selection marker gene incorporated in the binary vector. The hpt selection marker gene is operatively under the control of a pair of Pnos promoter and Nos terminator. Other pairs of promoter and terminator that can drive selection marker gene expression also are suitable for the purpose.

VI.B. Other Plant Transformation Vectors

Transformation without the use of Agrobacterium tumefaciens circumvents the requirement for T-DNA sequences in the chosen transformation vector and consequently vectors lacking these sequences can be utilized in addition to vectors such as the ones described above which contain T-DNA sequences. Transformation techniques that do not rely on Agrobacterium include transformation via particle bombardment, protoplast uptake (e.g. PEG and electroporation) and microinjection. The choice of vector can depend on the technique chosen for the species being transformed. Below, the construction of typical vectors suitable for non-Agrobacterium transformation is described.

pCIB3064. pCIB3064 is a pUC-derived vector suitable for direct gene transfer techniques in combination with selection by the herbicide basta (or phosphinothricin). The plasmid pCIB246 comprises the CaMV ³⁵S promoter in operational fusion to the E. coli GUS gene and the CaMV ³⁵S transcriptional terminator and is described in PCT International Publication No. WO 93/07278. The ³⁵S promoter of this vector contains two ATG sequences 5′ of the start site. These sites are mutated using standard PCR techniques in such a way as to remove the ATGs and generate the restriction sites Sspl and PvuII. The new restriction sites are 96 and 37 bp away from the unique SalI site and 101 and 42 bp away from the actual start site. The resultant derivative of pCIB246 is designated pCIB3025.

The GUS gene is then excised from pCIB3025 by digestion with SalI and SacI, the termini rendered blunt and religated to generate plasmid pCIB3060. The plasmid pJIT82 is obtained from the John Innes Centre (Norwich, United Kingdom), and a 400 bp SmaI fragment containing the bar gene from Streptomyces viridochromogenes is excised and inserted into the HpaI site of pCIB3060 (Thompson et al., 1987). This generated pCIB3064, which comprises the bar gene under the control of the CaMV ³⁵S promoter and terminator for herbicide selection, a gene for ampicillin resistance (for selection in E. coli) and a polylinker with the unique sites SphI, PstI, HindIII, and BamHI. This vector is suitable for the cloning of plant expression cassettes containing their own regulatory signals.

pSOG19 and PSOG35. pSOG35 is a transformation vector that utilizes the E. coli gene dihydrofolate reductase (DHFR) as a selectable marker conferring resistance to methotrexate. PCR is used to amplify the ³⁵S promoter (−800 bp), intron 6 from the maize Adh1 gene (−550 bp) and 18 bp of the GUS untranslated leader sequence from pSOG10. A 250-bp fragment encoding the E. coli dihydrofolate reductase type II gene is also amplified by PCR and these two PCR fragments are assembled with a SacI-PstI fragment from pB1221 (Clontech, Palo Alto, Calif., United States of America) that comprises the pUC19 vector backbone and the nopaline synthase terminator. Assembly of these fragments generates pSOG19 which contains the 35S promoter in fusion with the intron 6 sequence, the GUS leader, the DHFR gene and the nopaline synthase terminator. Replacement of the GUS leader in pSOG19 with the leader sequence from Maize Chlorotic Mottle Virus (MCMV) generates the vector pSOG35. pSOG19 and pSOG35 carry a β-lactamase gene from the pUC vector for ampicillin resistance and have HindIII, SphI, PstI and EcoRI sites available for the cloning of foreign substances.

VI.C. Selectable Markers

For certain target species, different antibiotic or herbicide selection markers can be preferred. Selection markers used routinely in transformation include the nptil gene, which confers resistance to kanamycin and related antibiotics (Messing & Vierra, 1982; Bevan et al., 1983), the bar gene, which confers resistance to the herbicide phosphinothricin (White et al., 1990; Spencer et al., 1990), the hph gene, which confers resistance to the antibiotic hygromycin (Blochlinger & Diggelmann, 1984), the dhfr gene, which confers resistance to methotrexate (Bourouis & Jarry, 1983), and the EPSP synthase gene, which confers resistance to glyphosate (U.S. Pat. Nos. 4,940,935 and 5,188,642).

VII. Transformation

The presently disclosed subject matter also provides a method for stably modulating expression of a gene in a plant. In one embodiment, the method comprises (a) transforming a plurality of plant cells to create a plurality of transformed plant cells, wherein the transformed plants cells have been transformed with an Agrobacterium tumefaciens binary vector comprising (i) a nucleic acid sequence encoding a selectable marker; and (ii) a nucleic acid sequence encoding a short interfering RNA (siRNA) operatively linked to a promoter and a transcription termination sequence; (b) treating the plant cells with a drug under conditions sufficient to kill those plant cells that did not receive the binary vector, wherein the selectable marker provides resistance to the drug; (c) growing the transformed plant cells under conditions sufficient to select for those transformed plant cells that have integrated the binary vector into their genomes; (d) screening the plurality of transformed plant cells for expression of the siRNA encoded by the expression vector; (e) selecting a plant cell that expresses the siRNA; and (f) regenerating the plant from the plant cell that expresses the siRNA, whereby expression of the gene in the plant is stably modulated.

The presently disclosed subject matter is based on the introduction of a stable and heritable siRNA into plant cells to specifically manipulate a gene of the interest. As disclosed herein, this concept has been demonstrated through Agrobacterium transformation, bout would also be applicable to other approaches for transformation, such as bombardment. Thus, it should be understood that the mechanism of transformation of a plant cell is not limited to the Agrobacterium-mediated techniques disclosed in certain embodiments herein. Any transformation technique that results in stable expression of a nucleic acid (for example, an siRNA) of the presently disclosed subject matter can be employed with the methods disclosed herein.

In one embodiment, the presently disclosed subject matter provides vectors for the stable transformation of plants.

Once a nucleic acid sequence of the presently disclosed subject matter has been cloned into an expression system, it is transformed into a plant cell. The receptor and target expression cassettes of the presently disclosed subject matter can be introduced into the plant cell in a number of art-recognized ways. Methods for regeneration of plants are also well known in the art. For example, Ti plasmid vectors have been utilized for the delivery of foreign DNA, as have direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

VII.A. Transformation of Dicotyledons

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation-mediated uptake, particle bombardment-mediated delivery, or microinjection. Examples of these techniques are disclosed in Paszkowski et al., 1984; Potrykus et al., 1985; Reich et al., 1986; and Klein et al., 1987. In each case the transformed cells are regenerated to whole plants using standard techniques known in the art.

Agrobacterium-mediated transformation is a useful technique for transformation of dicotyledons because of its high efficiency of transformation and its broad utility with many different species. Agrobacterium transformation typically involves the transfer of the binary vector carrying the foreign DNA of interest (e.g. pSIT) to an appropriate Agrobacterium strain that can depend on the complement of vir genes carried by the host Agrobacterium strain either on a co-resident Ti plasmid or chromosomally (e.g. strain C58 or strains pCIB542 for pCIB200 and pCIB2001; Uknes et al., 1993). The transfer of the recombinant binary vector to Agrobacterium is accomplished by a triparental mating procedure using E. coli carrying the recombinant binary vector, a helper E. coli strain that carries a plasmid such as pRK2013 and which is able to mobilize the recombinant binary vector to the target Agrobacterium strain. Alternatively, the recombinant binary vector can be transferred to Agrobacterium by DNA transformation (Höfgen & Willmitzer, 1988).

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050; 5,036,006; and 5,100,792; all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium, or a bacteriophage, each containing DNA sought to be introduced) can also be propelled into plant cell tissue.

VII.B. Transformation of Monocotyledons

Transformation of most monocotyledon species has now also become routine. Exemplary techniques include direct gene transfer into protoplasts using PEG or electroporation, and particle bombardment into callus tissue. Transformations can be undertaken with a single DNA species or multiple DNA species (i.e., co-transformation), and both these techniques are suitable for use with the presently disclosed subject matter. Co-transformation can have the advantage of avoiding complete vector construction and of generating transgenic plants with unlinked loci for the gene of interest and a selectable marker, enabling the removal of the selectable marker in subsequent generations, should this be regarded as desirable. However, a disadvantage of the use of co-transformation is the less than 100% frequency with which separate DNA species are integrated into the genome (Schocher et al., 1986).

Patent Applications EP 0 292 435, EP 0 392 225, and WO 93/07278 describe techniques for the preparation of callus and protoplasts from an elite inbred line of maize, transformation of protoplasts using PEG or electroporation, and the regeneration of maize plants from transformed protoplasts. Gordon-Kamm et al., 1990 and Fromm et al., 1990 have published techniques for transformation of A188-derived maize line using particle bombardment. Furthermore, WO 93/07278 and Koziel et al., 1993 describe techniques for the transformation of elite inbred lines of maize by particle bombardment. This technique utilizes immature maize embryos of 1.5-2.5 mm length excised from a maize ear 14-15 days after pollination and a PDS-1000He Biolistic particle delivery device (DuPont Biotechnology, Wilmington, Del., United States of America) for bombardment.

Transformation of rice can also be undertaken by direct gene transfer techniques utilizing protoplasts or particle bombardment. Protoplast-mediated transformation has been disclosed for Japonica-types and Indica-types (Zhang et al., 1988; Shimamoto et al., 1989; Datta et al., 1990). Both types are also routinely transformable using particle bombardment (Christou et al., 1991). Furthermore, WO93/21335 describes techniques for the transformation of rice via electroporation.

Patent Application EP 0 332 581 describes techniques for the generation, transformation, and regeneration of Pooideae protoplasts. These techniques allow the transformation of Dactylis and wheat. Furthermore, wheat transformation has been disclosed in Vasil et al., 1992 using particle bombardment into cells of type C long-term regenerable callus, and also by Vasil et al., 1993 and Weeks et al., 1993 using particle bombardment of immature embryos and immature embryo-derived callus.

A representative technique for wheat transformation, however, involves the transformation of wheat by particle bombardment of immature embryos and includes either a high sucrose or a high maltose step prior to gene delivery. Prior to bombardment, embryos (0.75-1 mm in length) are plated onto MS medium with 3% sucrose (Murashige & Skoog, 1962) and 3 mg/l 2,4-dichlorophenoxyacetic acid (2,4-D) for induction of somatic embryos, which is allowed to proceed in the dark. On the chosen day of bombardment, embryos are removed from the induction medium and placed onto the osmoticum (i.e., induction medium with sucrose or maltose added at the desired concentration, typically 15%). The embryos are allowed to plasmolyze for 2-3 hours and are then bombarded. Twenty embryos per target plate are typical, although not critical. An appropriate gene-carrying plasmid (such as pCIB3064 or pSG35) is precipitated onto micrometer size gold particles using standard procedures. Each plate of embryos is shot with the DuPont BIOLISTICS® helium device using a burst pressure of about 1000 pounds per square inch (psi) using a standard 80 mesh screen. After bombardment, the embryos are placed back into the dark to recover for about 24 hours (still on osmoticum). After 24 hours, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l BASTA® in the case of pCIB3064 and 2 mg/l methotrexate in the case of pSOG35). After approximately one month, developed shoots are transferred to larger sterile containers known as “GA7s” which contain half-strength MS, 2% sucrose, and the same concentration of selection agent.

Transformation of monocotyledons using Agrobacterium has also been disclosed. See WO 94/00977 and U.S. Pat. No. 5,591,616, both of which are incorporated herein by reference. See also Negrotto et al., 2000, incorporated herein by reference. Like other Agrobacterium-mediated binary vector system used for the transformation of monocotyledons, pSIT can also be employed to modify monocotyledons.

VII.C. Transformation of Plastids

Seeds of Nicotiana tabacum c.v. ‘Xanthi nc’ are germinated seven per plate in a 1″ circular array on T agar medium and bombarded 12-14 days after sowing with 1 μm tungsten particles (M10, Biorad, Hercules, Calif., United States of America) coated with DNA from representative plasmids essentially as disclosed (Svab & Maliga, 1993). Bombarded seedlings are incubated on T medium for two days after which leaves are excised and placed abaxial side up in bright light (350-500 pmol photons/m²/s) on plates of RMOP medium (Svab et al., 1990) containing 500 μg/ml spectinomycin dihydrochloride (Sigma, St. Louis, Mo., United States of America). Resistant shoots appearing underneath the bleached leaves three to eight weeks after bombardment are subcloned onto the same selective medium, allowed to form callus, and secondary shoots isolated and subcloned. Complete segregation of transformed plastid genome copies (homoplasmicity) in independent subclones is assessed by standard techniques of Southern blotting (Sambrook & Russell, 2001). BamHI/EcoRI-digested total cellular DNA (Mettler, 1987) is separated on 1% Tris-borate-EDTA (TBE) agarose gels, transferred to nylon membranes (Amersham Biosciences, Piscataway, N.J., United States of America) and probed with ³²P-labeled random primed DNA sequences corresponding to a 0.7 kb BamHI/HindIII DNA fragment from pC8 containing a portion of the rps7/12 plastid targeting sequence. Homoplasmic shoots are rooted aseptically on spectinomycin-containing MS/IBA medium (McBride et al., 1994) and transferred to the greenhouse.

VIII. Plants, Breeding, and Seed Production

VII.A. Plants

The presently disclosed subject matter also provides plants comprising the disclosed compositions. In one embodiment, the plant is characterized by a modification of a phenotype or measurable characteristic of the plant, the modification being attributable to the expression cassette. In one embodiment, the modification involves, for example, nutritional enhancement, increased nutrient uptake efficiency, enhanced production of endogenous compounds, or production of heterologous compounds. In another embodiment, the modification includes having increased or decreased resistance to a herbicide, environmental stress, or a pathogen. In another embodiment, the modification includes having enhanced or diminished requirement for light, water, nitrogen, or trace elements. In another embodiment, the modification includes being enriched for an essential amino acid as a proportion of a polypeptide fraction of the plant. In another embodiment, the polypeptide fraction can be, for example, total seed polypeptide, soluble polypeptide, insoluble polypeptide, water-extractable polypeptide, and lipid-associated polypeptide. In another embodiment, the modification includes overexpression, underexpression, antisense modulation, sense suppression, inducible expression, inducible repression, or inducible modulation of a gene. In alternative embodiments, the modifications can include decreased or increased lignin content, lignin composition and/or structure changes, decreased or increased cellulose content, crystallinity and DP (degree of polymerization) changes, fiber property and morphology modifications, and/or increased resistance to pathogens, common diseases, and environment stresses in a tree.

VIII.B. Breeding

The plants obtained via transformation with a nucleic acid sequence of the presently disclosed subject matter can be any of a wide variety of plant species, including monocots and dicots, and angiosperms and gymnosperms; however, the plants used in the method for the presently disclosed subject matter are selected in one embodiment from the list of agronomically important target crops set forth hereinabove. The modification of expression of a gene in accordance with the presently disclosed subject matter in combination with other characteristics important for production and quality can be incorporated into plant lines through breeding. Breeding approaches and techniques are known in the art. See e.g., Welsh, 1981; Wood, 1983; Mayo, 1987; Singh, 1986; Wricke & Weber, 1986.

The genetic properties engineered into the transgenic seeds and plants disclosed above are passed on by sexual reproduction or vegetative growth and can thus be maintained and propagated in progeny plants. Generally, maintenance and propagation make use of known agricultural methods developed to fit specific purposes such as tilling, sowing, or harvesting. Specialized processes such as hydroponics or greenhouse technologies can also be applied. As the growing crop is vulnerable to attack and damage caused by insects or infections as well as to competition by weed plants, measures are undertaken to control weeds, plant diseases, insects, nematodes, and other adverse conditions to improve yield. These include mechanical measures such as tillage of the soil or removal of weeds and infected plants, as well as the application of agrochemicals such as herbicides, fungicides, gametocides, nematicides, growth regulants, ripening agents, and insecticides.

Use of the advantageous genetic properties of the transgenic plants and seeds according to the presently disclosed subject matter can further be made in plant breeding, which aims at the development of plants with improved properties such as tolerance of pests, herbicides, or abiotic stress, improved nutritional value, increased yield, or improved structure causing less loss from lodging or shattering. The various breeding steps are characterized by well-defined human intervention such as selecting the lines to be crossed, directing pollination of the parental lines, or selecting appropriate progeny plants.

Depending on the desired properties, different breeding measures are taken. The relevant techniques are well known in the art and include, but are not limited to, hybridization, inbreeding, backcross breeding, multiline breeding, variety blend, interspecific hybridization, aneuploid techniques, etc. Hybridization techniques can also include the sterilization of plants to yield male or female sterile plants by mechanical, chemical, or biochemical means. Cross-pollination of a male sterile plant with pollen of a different line assures that the genome of the male sterile but female fertile plant will uniformly obtain properties of both parental lines. Thus, the transgenic seeds and plants according to the presently disclosed subject matter can be used for the breeding of improved plant lines that, for example, increase the effectiveness of conventional methods such as herbicide or pesticide treatment or allow one to dispense with said methods due to their modified genetic properties. Alternatively new crops with improved stress tolerance can be obtained, which, due to their optimized genetic “equipment”, yield harvested product of better quality than products that were not able to tolerate comparable adverse developmental conditions (for example, drought).

VIII.C. Seed Production

Embodiments of the presently disclosed subject matter also provide seed from plants modified using the disclosed methods.

In seed production, germination quality and uniformity of seeds are essential product characteristics. As it is difficult to keep a crop free from other crop and weed seeds, to control seedborne diseases, and to produce seed with good germination, fairly extensive and well-defined seed production practices have been developed by seed producers who are experienced in the art of growing, conditioning, and marketing of pure seed. Thus, it is common practice for the farmer to buy certified seed meeting specific quality standards instead of using seed harvested from his own crop. Propagation material to be used as seeds is customarily treated with a protectant coating comprising herbicides, insecticides, fungicides, bactericides, nematicides, molluscicides, or mixtures thereof. Customarily used protectant coatings comprise compounds such as captan, carboxin, thiram (tetramethylthiuram disulfide; TMTD®; available from R. T. Vanderbilt Company, Inc., Norwalk, Conn., United States of America), methalaxyl (APRON XL®; available from Syngenta Corp., Wilmington, Del., United States of America), and pirimiphos-methyl (ACTELLIC®; available from Agriliance, LLC, St. Paul, Minn., United States of America). If desired, these compounds are formulated together with further carriers, surfactants, and/or application-promoting adjuvants customarily employed in the art of formulation to provide protection against damage caused by bacterial, fungal, or animal pests. The protectant coatings can be applied by impregnating propagation material with a liquid formulation or by coating with a combined wet or dry formulation. Other methods of application are also possible such as treatment directed at the buds or the fruit.

IX. Transgenic Plants

A “transgenic plant” is one that has been genetically modified to contain and express an siRNA. A transgenic plant can be genetically modified to contain and express at least one homologous or heterologous DNA sequence operatively linked to and under the regulatory control of transcriptional control sequences which function in plant cells or tissue or in whole plants. As used herein, a transgenic plant also refers to progeny of the initial transgenic plant where those progeny contain and are capable of expressing the homologous or heterologous coding sequence under the regulatory control of the plant-expressible transcription control sequences described herein. Seeds containing transgenic embryos are encompassed within this definition as are cuttings and other plant materials for vegetative propagation of a transgenic plant.

When plant expression of a homologous or heterologous gene or coding sequence of interest is desired, that coding sequence is operatively linked in the sense orientation to a suitable promoter and advantageously under the regulatory control of DNA sequences which quantitatively regulate transcription of a downstream sequence in plant cells or tissue or in planta, in the same orientation as the promoter, so that a sense (i.e., functional for translational expression) mRNA is produced. A transcription termination signal, for example, as polyadenylation signal, functional in a plant cell is advantageously placed downstream of an siRNA-encoding sequence, and a selectable marker which can be expressed in a plant, can be covalently linked to the inducible expression unit so that after this DNA molecule is introduced into a plant cell or tissue, its presence can be selected and plant cells or tissue not so transformed will be killed or prevented from growing.

Where tissue specific expression of the plant-expressible siRNA coding sequence is desired, the skilled artisan will choose from a number of well-known sequences to mediate that form of gene expression as disclosed herein. Environmentally regulated promoters are also well known in the art, and the skilled artisan can choose from well-known transcription regulatory sequences to achieve the desired result.

Summarily, the presently disclosed subject matter can be applied to, among other applications, the following:
1. Specifically silence target gene in a stable and heritable manner;
2. Enhance target gene function;
3. Regulate transcriptional activity of target promoter; and
4. Molecular operation through siRNA-induced silencing signal movement.

### EXAMPLES

The following Examples have been included to illustrate modes of the presently disclosed subject matter. Certain aspects of the following Examples are described in terms of techniques and procedures found or contemplated by the present co-inventors to work well in the practice of the presently disclosed subject matter. These Examples illustrate standard laboratory practices of the co-inventors. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Identification of Potential siRNA Target Sites in Any RNA Sequence

The sequence of an RNA target of interest, such as a human mRNA transcript, is screened for target sites, for example by using a computer-based folding algorithm. In a non-limiting example, the sequence of a gene or RNA gene transcript derived from a database, such as GENBANK®, is used to generate siRNA targets having complementarity to the target. Such sequences can be obtained from a database, or can be determined experimentally as known in the art. Target sites that are known, for example, those target sites determined to be effective target sites based on studies with other nucleic acid molecules, for example ribozymes or antisense, or those targets known to be associated with a disease or condition such as those sites containing mutations or deletions, can be used to design siRNA molecules targeting those sites as well. Various parameters can be used to determine which sites are the most suitable target sites within the target RNA sequence. These parameters include but are not limited to secondary or tertiary RNA structure, the nucleotide base composition of the target sequence, the degree of homology between various regions of the target sequence, or the relative position of the target sequence within the RNA transcript. Based on these determinations, any number of target sites within the RNA transcript can be chosen to screen siRNA molecules for efficacy, for example by using in vitro RNA cleavage assays, cell culture, or animal models. In a non-limiting example, anywhere from 1 to 1000 target sites are chosen within the transcript based on the size of the siRNA construct to be used. High throughput screening assays can be developed for screening siRNA molecules using methods known in the art, such as with multi-well or multi-plate assays to determine efficient reduction in target gene expression.

### Example 2

### Design of siRNAs Directed Against the GUS Gene

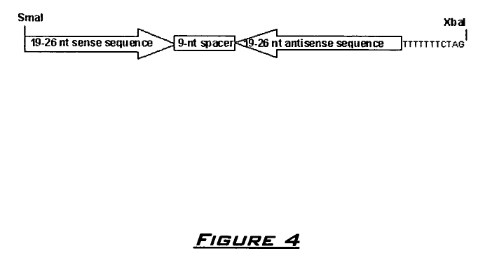

Based on the standard design rules (Elbashir et al., 2002) two 19 nt sequences (designated GT1 and GT2) targeting two distinct sites in the GUS mRNA were selected for constructing the expression vectors. Individual siRNA templates comprised the 19 nt fragment linked via a 9 nt spacer (see e.g., FIG. 4) to the reverse complement of the same 19 nt sequence. Each template was cloned into a vector comprising a human H1 RNA transcription unit under the control of its cognate gene promoter (FIG. 5). The resulting transcript was predicted to adopt an inverted hairpin RNA structure containing one (for GT1) or two (for GT2) 3′ overhanging uridines, giving rise to siRNA-like transcripts adopting GT1 or GT2 sequences (FIG. 5). As shown in FIG. 5, GT1 produces an siRNA-like transcript comprising SEQ ID NO: 14—9 nt spacer—SEQ ID NO: 15 (bottom left), and GT2 produces a transcript comprising SEQ ID NO 16—9 nt spacer—SEQ ID NO: 17.

### Example 3

### RNA Silencing with Human H1 Promoter-Containing Constructs

Agrobaterium tumefaciens C58 cells were transformed with the GT1 and GT2 vectors and used to transform a transgenic tobacco line expressing a GUS transgene (Hu et al., 1998). To transfer tobacco, GUS containing tobacco leaf disks were infected with Agrobacteria C58 strain harboring siRNA construct. Transformants were selected on MS104 containing 25 mg/L hygromycin and 300 mg/L claforan. The hygromycin-resistant shoots were placed on hormone free MSO agar medium containing 25 mg/L hygromycin and 300 mg/L claforan for root regeneration, and transgenic tobacco seedlings were planted to soil growing in a greenhouse.

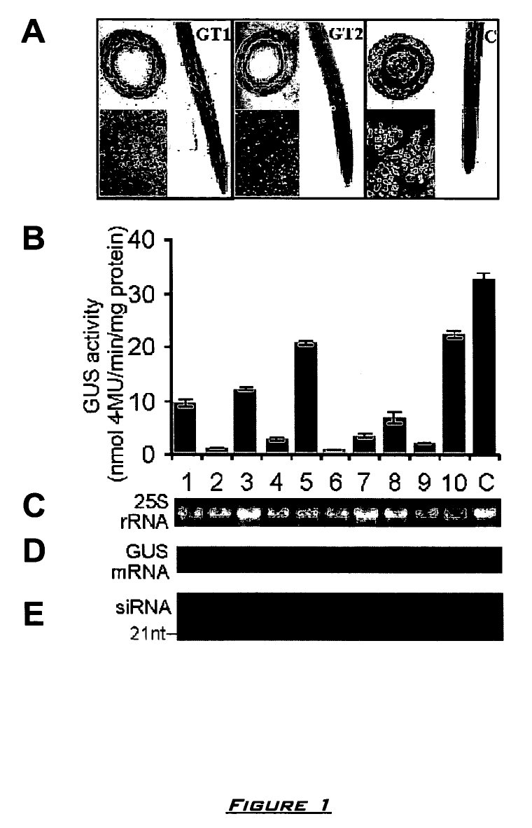

Twenty-three transgenic plants were produced from the GT1 construct and nineteen from GT2. Transgenic plants and GUS-carrying control plants were characterized at about one month old. The stem, leaf, and root of a majority of the GT1 and GT2 transgenics exhibited either reduced or no GUS staining (FIG. 1A). Assays of GUS protein activity in leaves indicated that 74% of the GT1 transgenics had a reduction in GUS activity ranging from 12 to 94%, and 84% of the GT2 transgenics exhibited 31 to 97% GUS activity reduction. The reduction in GUS activity (see FIG. 1B) reflected diminished GUS mRNA levels in these plants (see FIGS. 1C and 1D). A small discrete RNA of about 21 nt in length was present in the transgenic lines having reduced GUS mRNA and protein activity, but absent from the control line (see FIG. 1E). Overall, the abundance of this 21 nt RNA was inversely correlated with the abundance of GUS mRNA in these plants (see FIGS. 1C and 1E).

The gene silencing efficiency appeared to be independent of the GUS mRNA target sites and of the number of uridine residues (1 vs. 2) in the engineered siRNA transcripts. Furthermore, the silencing effect remained in about 90% of the T₁ plants analyzed.

### Example 4

### Cloning of the Arabidopsis 7SL4 Promoter

Two oligonucleotides corresponding to the promoter region of the Arabidopsis thaliana At7SL4 gene were designed based upon data present in the publicly available Arabidopsis database (available at through website of The Institute for Genomic Research). These primers are SLpF (5′-GGAATTCTGCGTTTGMGAAGAGTGTTTGA-3′; SEQ ID NO: 1) as the forward primer (with the addition of an EcoRI site at the 5′ end) and SLpR (5′-GCCCGGGMGATCGGTTCGTGTMTATAT-3′; SEQ ID NO: 2) as the reverse primer (with addition of a SmaI site at the 5′ end). These two primers flank the At7SL4 gene promoter at both ends and were used for PCR amplification of the promoter fragment from Arabidopsis thaliana (Columbia ecotype) genomic DNA.

The PCR product amplified from Arabidopsis genomic DNA using primers SLpF and SLpR was cloned into the PCR®2.1-TOPO® system (Invitrogen Corp., Carlsbad, Calif., United States of America) and the sequence of the promoter fragment confirmed by sequencing. The resulting At7SL4 promoter clone was named pCRSLp7, and contained the following At7SL4 promoter sequence: GGMTTCTGCGTTTGAAGAAGAGTGTTTGATGTTCTCMGTMGTGAGT CTTATTGGGMTMTATTMCTCATGTTCTTCTTGCATTTGATTTCTTTGC CGCTCTCTTCTTCTATCTCAAATCTGTCTCTTCAATTTCACAGTTGGGCT TTTTATTAGTCTATMTGGGACTCAAMATAAGGCTTTGGCCCACATCAAA AAGATAAGTCAAATGAAAACTAAATTCAGTCTTTTGTCCCACATCGATCA CTCTACTCGTTTTGTGTTTGTTTATATATTACACGMCCGATCTTCCCGG GC (SEQ ID NO: 3). The sequences of the SLpF and SLpR primers are underlined.

### Example 5

### Cloning of the Arabidopsis At7SL4 Gene 3′ Non-translated Sequence

To clone the 3′-NTS of the At7SL4 gene, two oligos were synthesized based on sequence information available in the the Arabidopsis database as described hereinabove. The primers used were as follows: SLtF 5′-GTCTAGATTTTGATTTTGTTTTCCAAAACTTTCTACG-3′ (SEQ ID NO: 4),
was used as the forward primer (adds an XbaI site added to the 5′ end of the 3′-NTS); and SLtR 5′-GAAGCTTGGTGTTGATCACMCGATACA-3′ (SEQ ID NO: 5) was used as the reverse primer (adds a HindIII site to the 3′ end of the 3′-NTS). Using these two primers and Arabidopsis thaliana (Columbia ecotype) genomic DNA, PCR was employed to amplify a nucleic acid molecule comprising the 3′-NTS. The amplified nucleic acid molecule was cloned into the PCR®2.1-TOPO® system (Invitrogen Corp.) and sequenced (plasmid referred to herein as pCRSLt2). The correct At7SL4-3′-NTS nucleotide sequence was determined to be: GTCTAGATTTTGATTTTGTTTTCCAAAACTTTCTACGCTTTTTGTTTTTGG GTTTMTGCTTTMGAGGGMCAAAAACAAAGCTGTGAAAACTGAAAGC AAACTTTGMCAAAGCMGAGACTTMGAGTTGTATTTACAGCTTTTGTT CGATGTATGGAAATGTACAATTTTTTTGCTACTCAAAGAAATGAGACTTA AGAGTCAACGTTAAAAGAGCCAGGAGTAAAATGTCTAGGTATGATCTCA ATTGTATCGTTGTGATCMCACCMGCTTC (SEQ ID NO: 6). The sequences of the SLtF and SLtR primers are underlined.

### Example 6

Assembly of the siRNA Delivery Cassette

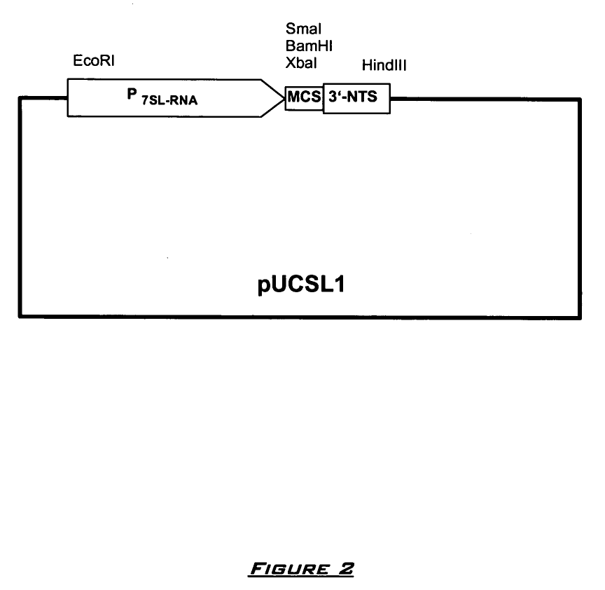

The 7SL4-RNA promoter sequence was released from pCRSLp7 by digestion with EcoRI and SmaI and then inserted into a pUC19 vector at the EcoRI and SmaI cloning sites, yielding a plasmid referred to herein as pUCSLp7-1. To assemble the siRNA delivery cassette including the elements of the 7SL4-RNA promoter and the 3′-NTS fragment, the At7SL4-3′-NTS sequence was released from pCRSLt2 by digestion with XbaI and HindIII. The At7SL4-3′-NTS sequence was thereafter ligated into the XbaI and HindIII cloning sites of pUCSLp7-1 to produce a construct named pUCSL1. This construct contained the siRNA delivery cassette in a pUC19 backbone vector. The siRNA expression cassette contains the At7SL4 promoter sequence and the At7SL4-3′-NTS sequence. Between these two elements is a multiple cloning site (MCS) including sites for SmaI, BamHI, and XbaI for insertion of target sequences (see FIG. 2).

### Example 7

### Plant 7SL Promoter-mediated siRNA Silencing of GUS Expression in Transgenic Tobacco

A plant promoter-based system was also tested. DNA-dependent RNA polymerase III 7SL RNA genes from Arabidopsis thaliana were employed, because the transcription of these small genes is controlled exclusively by their upstream external regulatory sequence elements (USE and TATA) and terminates at a run of five to seven thymidines. These features allowed for the incorporation of these sequences into expression vectors to efficiently produce siRNA duplexes that contained three to four 3′ overhanging uridines. From an A. thaliana At7SL4, the promoter and 3′-NTS region were cloned by PCR amplification: To clone the promoter region of the At7SL4 gene, PCR was carried out by using SLpF (5′-GGAATTCTGCGTTTGAAGMGAGTGTTTGA-3′; SEQ ID NO: 1) as a forward primer and SLpR (5′-GCCCGGGMGATCGGTTCGTGTMTATAT-3′; SEQ ID NO: 2) as a reverse primer and using Arabidopsis thaliana (Columbia ecotype) genomic DNA as a template. The PCR product was cloned into pCR2.1-TOPO vector (Invitrogen) and sequenced. The plasmid with correct At7SL promoter sequence was named pCRSLp7. The promoter region was then released from pCRSLp7 by digesting with Eco RI and Sma I and inserted into Eco RI-SmaI gap of pUC19. The resulting plasmid was named pUCSLp7-1.

The 3-NTR region of At7SL4 gene was amplified by PCR using SLtF (5′-GTCTAGATTTTGATTTTGTTTTCCAAAACTTTCTACG-3′; SEQ ID NO: 4) as a forward primer and SLtR (5′-GAAGCTTGGTGTTGATCACMCGATACA-3′; SEQ ID NO: 5) as a reverse primer and using Arabidopsis thaliana (Columbia ecotype) genomic DNA as a template. The PCR product was cloned into pCR2.1-TOPO vector (Invitrogen) and sequenced. The plasmid with correct At7SL 3′-NTR sequence was named pCRSLt2. The 3′-NTR region was then released from pCRSLt2 by digesting with Xba I and Hind III and inserted into Xba 1-Hind III gap of pUCSLp7-1 to assemble the siRNA expression module. The resulting plasmid was named pUCSL1.

The siRNA expression module with 289 bp of At7SL promoter region and 267 bp of At7SL 3′-NTS region was subsequently subcloned into pGPTV-HPT to replace the uidA-pAnos fragment. The resulting plasmid was named as pGPSL1.

The promoter, including a 289 bp fragment containing USE and TATA elements and a 3′-non-transcribed sequence (NTS) of 267 bp, was isolated. The promoter and NTS were cloned into pUC19 to assemble the siRNA expression vector (pUCSL1; see FIG. 2).

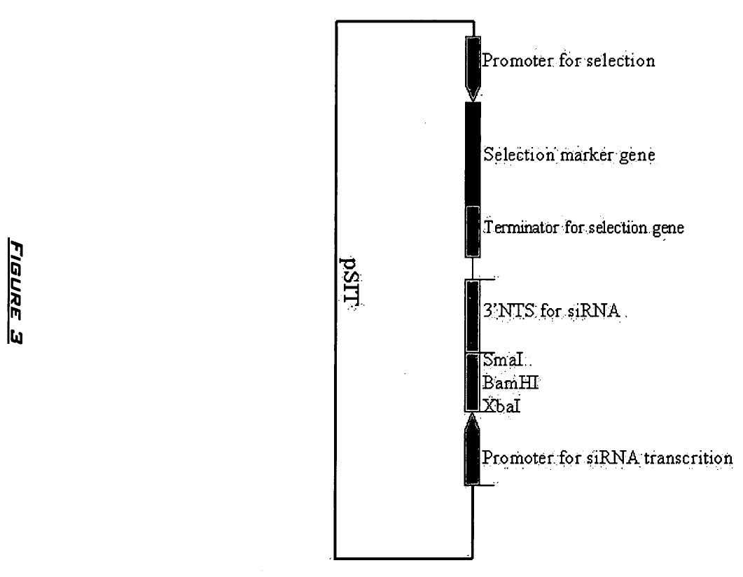

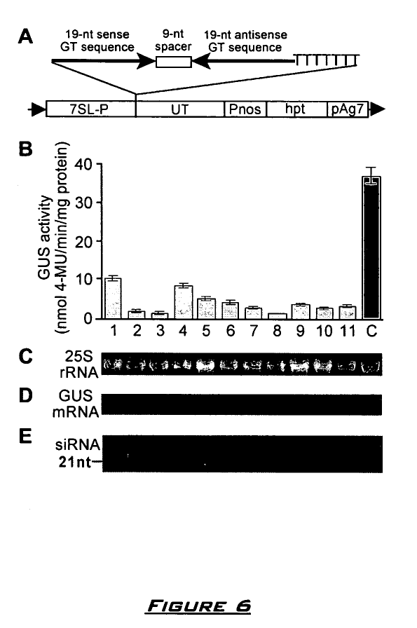

In addition to the GT1 and GT2 sequences described in Example 2, an additional 19 nt GUS mRNA sequence, referred to herein as GT3, was selected for constructing an additional siRNA template, following the general design described above in Example 2. siRNA templates corresponding to GT1, GT2, and GT3 were cloned into the pSIT expression vector (see FIGS. 3 and 6), which was then mobilized into A. tumefaciens C58 cells for transforming the transgenic GUS tobacco line described above in Example 2 (see also Hu et al., 1998). A total of 89 plants were produced containing one of these three expression constructs.

The same analysis schemes described in Example 2 were employed to screen transgenic plants. It was determined that 83% of these transgenic plants exhibited a reduction in GUS enzyme activity ranging from 20 to 99%. No apparent difference in overall GUS activity reduction efficiency was observed among these three expression constructs. The observed reduction in GUS enzyme activity correlated with diminished GUS mRNA level, and with the appearance/abundance of GUS-specific siRNAs. Together, these results validated a plant promoter-based siRNA gene silencing system.

### Discussion of Examples 1-7

A promoter fragment (289 bp) containing USE and TATA elements (7SL-P) and a 3′-NTS region (267 bp) of Arabidopsis At7SL4 gene was cloned into pUC19, from which the 7SL-P/NTS construct was excised and cloned into the pGPTV-HPT vector to replace the pAnos-uidA fragment. The resulting vector was named pGPSL-HPT, which contained an hpt selectable marker gene. GUS gene specific GT1, GT2, and GT3 (nt 81-99) sequence cassettes for the generation of the corresponding hairpin siRNAs (see FIG. 5), all containing a termination signal of seven thymidines, was inserted between the 7SL-P and 3′-NTS fragments in pGPSL-HPT. The resulting plasmid was then mobilized into A. tumefaciens C58 for transforming tobacco. Transgenic plants were analyzed according to the criteria described in Example 2.

Two effective siRNA systems for silencing a GUS reporter gene in a model plant have been described. For each system, it was demonstrated that siRNAs targeted to a number of distinct sites in the GUS mRNA resulted in a similar gene silencing effect. For both systems, reductions in mRNA levels were consistent with diminished protein levels, suggesting that gene silencing occurred through specific RNA interference rather than translational attenuation. The effectiveness of the human H1 promoter to induce gene silencing in a plant background indicated that the plant pol III complex was capable of initiating transcription from the mammalian pol III gene sequences, and that the machinery for processing cellular RNA species might be conserved in eukaryotic organisms.

The current results further showed that the two promoter systems are equally effective in inducing gene silencing in a plant background. Because these systems are incorporated with Agrobacterium-mediated gene transfer and whole plant regeneration, the observed gene silencing effect is expected to be persistent and inherited, rather than transient and unstable. With the availability of the genome sequence of several plant species, these siRNA vector systems can help invigorate a genome-wide analysis of gene function with specificity and efficacy and, thus, can be used to provide more precise mechanisms to generate agriculture crops with specific traits of economic importance.

### Example 8

### pSIT System for Stable Transformation of Plants

In order to introduce stably expressed siRNAs to plant tissues, a binary vector transformation system mediated by Agrobacterium was developed. The binary vector construct contained an siRNA delivery cassette and a selectable marker gene under the control of separate promoters, and is referred to herein as pSIT (small interfering RNA transformation system). See FIG. 3. Cloning sites for SmaI, BamHI, and XbaI have been included in pSIT, and can be used for the insertion of target gene sequences in a structure designed to form a double-stranded RNA when the target gene sequences are transcribed. The insert structure is in one embodiment a 19 to 26-nucleotide sequence corresponding to the sense strand of a target gene followed by the complementary antisense sequence. The sense and antisense sequences are separated by a 9-nucleotide spacer (5′-TTCAGATGA-3′; see FIG. 4). At the 3′-end of the structure, a string of several thymidines (in one embodiment, a string of 7) was added to signal termination of transcription from the promoter.

### Example 9

### siRNA Targeting of the GUS gene in Transgenic Tobacco

Three siRNA expression constructs were designed to target the β-glucuronidase (GUS) gene. These expression constructs are referred to herein as pGSGT1, pGSGT2, and pGSGT3. Each of these expression constructs targets a different position of the GUS gene sequence. pGSGT1 targets nucleotides 80-98, pGSGT2 targets nucleotides 89-107, and pGSGT3 targets nucleotides 81-99 of the GUS coding region (GENBANK® Accession No. AY100472; SEQ ID NO: 13).

To produce a double-stranded DNA structure for cloning, a pair of oligonucleotides, one sense strand and one complementary strand, were synthesized according to each target sequence. For pGSGT1, the sense strand oligo had the sequence 5′-TACACTGTGGAATT GATCAGCGTTCAGATGACGCTGATCAATTCCACAGTTTTTTTT (SEQ ID NO: 7) and the complementary strand oligo had the sequence 5′-CTAGAAAAAAAACTGTGGMTTGATCAGCGTCATCTGMCGCTGATCM TTCCACAGTGTA (SEQ ID NO: 8). For pGSGT2, the sense strand oligo had the sequence 5′-TACTTGATCAGCGTTGGTGGGATTCAGATGATC CCACCMCGCTGATCMTTTTTTT (SEQ ID NO: 9) and the complementary strand oligo had the sequence 5′-CTAGAAAAAAT TGATCAGCGTTGGTGGGATCATCTGAATCCCACCMCGCTGATCMGTA (SEQ ID NO: 10). For pGSGT3, the sense strand oligo had the sequence 5′-TACCTGTGGAATTGATCAGCGTTTCAGATGMCGCTGATCMTTCCACA GTTTTTTT (SEQ ID NO: 11) and the complementary strand oligo had the sequence 5′-CTAGAMAAAACTGTGGMTTGATCAGCGTTCATCTGAAA CGCTGATCAATTCCACAGGTA (SEQ ID NO: 12).

After the sense and the corresponding complementary strands were annealed together, they formed a double-stranded DNA molecule that was inserted into the pSIT system via the cloning sites. In addition to the GT1 and GT2 sequences that were used in the H1 promoter construct, another 19 nt GUS mRNA sequence, named GT3, was selected for constructing the siRNA templates. After confirming the sequence of each inserted molecule by sequencing, the constructs were transformed into Agrobacterium and transformed into transgenic tobacco plants expressing GUS according to the method presented hereinabove. A total of 89 plants were regenerated from these three expression constructs under antibiotic selection. The results of transgenic plant analyses demonstrated that 83% of these transgenic plants exhibited a reduction in GUS enzyme activity, ranging from 20 to 99%. No apparent difference in overall GUS activity reduction efficiency was observed among these three expression constructs. Such a reduction correlated with diminished GUS mRNA level and with the appearance/abundance of the GUS-specific siRNAs. Together, these results validated a plant promoter-based siRNA gene silencing system.

Histochemical localization of GUS activity in stem hand-sections and leaf discs was conducted as described in Jefferson et al., 1987. To analyze the activity of GUS protein, about 100 mg of leaves from transgenic plants cultured in vitro were collected and frozen in liquid nitrogen. The leaves were ground in 800 μL GUS extraction buffer (50 mM phosphate buffer, pH 7.4, 10 mM DTT, 1 mM Na₂-EDTA, 0.1% sodium lauryl sarcosine, 0.1% Triton-X 100) using FASTPREP™ FP120 (Savant Instrument Inc., Holbrook, New York, United States of America). The GUS activity was analyzed as described (Jefferson et al., 1987). Fluorescence was detected by TD-700 Fluorometer (Turner Designs, Inc., Sunnyvale, Calif., United States of America). The protein concentration was analyzed using Protein Assay Dye Reagent Concentrate (Bio-Rad Laboratories, Hercules, Calif., United States of America) using a DU® 800 Spectrophotometer (Beckman Coulter, Inc., Fullerton, Calif., United States of America).

### Example 10

### siRNA-Based Gene Modulation in Trees

siRNA-based gene modification system can be used for modulating gene expression in trees. Representative, non-limiting genes the expression of which can be modulated include genes involved in the lignin and cellulose biochemical pathways. Moreover, the system is particularly useful for the manipulation of the genes which have multiple family members. Only a short sequence of the target gene is needed in the siRNA system, allowing the design of an siRNA target sequence to be highly specific and discernable from the other family member genes or other unknown genes which share a high sequence homology with the target member.

### Example 11

### Overexpression of a Gene by siRNA

When GUS tobacco plants were transformed with a GUS siRNA, a majority of the plants exhibited strong GUS silencing as discussed hereinabove. However, overexpression of GUS gene was also repeatedly observed in the plants transformed with 2 constructs under the control of an H1 promoter and 3 constructs driven by an 7SL promoter.

### References

The references listed below as well as all references cited in the specification are incorporated herein by reference to the extent that they supplement, explain, provide a background for or teach methodology, techniques, and/or compositions employed herein.
Adelman J P, Hayflick J S, Vasser M & Seeburg P H (1983) In Vitro Deletional Mutagenesis for Bacterial Production of the 20,000-Dalton Form of Human Pituitary Growth Hormone. DNA 2: 183-193.
Agrawal S (ed.) Methods in Molecular Biology, volume 20, Humana Press, Totowa, N.J., United States of America.
Altschul S F, Gish W, Miller W, Myers E W & Lipman D J (1990) Basic Local Alignment Search Tool. J Mol Biol 215: 403-410.
Anterola A M & Lewis N G (2002) Trends in lignin modification: a comprehensive analysis of the effects of genetic manipulations/mutations on lignification and vascular integrity. Phytochemistry 61: 221-94.
Ausubel F M, Brent R, Kingston R E, Moore D D, Seidman J G, Smith J A & Struhl K, eds (1989) Current Protocols in Molecular Biology. Wiley, New York, N.Y., United States of America.
Bartlett S G, Grossman A R & Chua N-H (1982) in Methods in Chloroplast Molecular Biology, (Edelman M, Hallick R B & Chua N-H, eds.) Elsevier Biomedical Press, New York, N.Y., United States of America, pp. 1081-1091.
Bass B L (2001) RNA interference: The short answer. Nature 411: 428-429.
Becker D, Kemper E, Schell J & Masterson R (1992) New plant binary vectors with selectable markers located proximal to the left T-DNA border. Plant Mol Biol 20: 1195-1197.
Bernstein E, Caudy A A, Hammond S M & Hannon G J (2001) Role for a bidentate ribonuclease in the initiation step of RNA interference. Nature 409: 363-366.
Bevan M (1984) Binary Agrobacterium vectors for plant transformation. Nucl. Acids Res 12: 8711-21.
Bevan M, Flavell R B & Chilton M D (1983) A chimeric antibiotic resistance gene as a selectable marker for plant cell transformation. Nature 304: 184-187.
Binet M N Weil J H & Tessier L H (1991) Structure and expression of sunflower ubiquitin genes. Plant Mol Biol 17: 395-407.
Blochinger K & Diggelmann H (1984) Hygromycin B phosphotransferase as a selectable marker for DNA transfer experiments with higher eucaryotic cells. Mol Cell Biol 4: 2929-2931.
Boerjan W, Ralph J & Baucher M (2003) Lignin biosynthesis. Annu Rev Plant Biol 54: 519-46.
Bourouis M & Jarry B (1983) Vectors containing a prokaryotic dihydrofolate reductase gene transform Drosophila cells to methotrexate-resistance. EMBO J. 2: 1099-1104.
Brummelkamp T R, Bernards R & Agami R (2002) A system for stable expression of short interfering RNAs in mammalian cells. Science 296: 550-553.
Callis J, Fromm M & Walbot V (1987) Introns increase gene expression in cultured maize cells. Genes Dev 1: 1183-1200.
Canadian Patent Application No. 2,359,180
Chibbar R N, Kartha K K, Datla R S S, Leung N, Caswell K, Mallard C S & Steinhauer L (1993) The effect of different promoter-sequences on transient expression of gus reporter gene in cultured barley (Hordeum vulgare L.) cells. Plant Cell Rep 12: 506-509.
Christensen A H & Quail P H (1989) Sequence analysis and transcriptional regulation by heat shock of polyubiquitin transcripts from maize. Plant Mol Biol 12: 619-632.
Christou P, Ford T & Kofron M (1991) Production of transgenic rice (Oryza sativa L.) plants from agronomically important indica and japonica varieties via electric discharge particle acceleration of exogenous DNA into immature zygotic embryos. Bio/Technology 9: 957-962.
Comai L, Larson-Kelly N, Kiser J, Mau C J, Pokalsky A R, Shewmaker C K, McBride K, Jones A & Stalker D M (1988) Chloroplast transport of a ribulose bisphosphate carboxylase small subunit-5-enolpyruvyl 3-phosphoshikimate synthase chimeric protein requires part of the mature small subunit in addition to the transit peptide. J Biol Chem 263: 15104-15109.
Datta S K, Peterhans A, Datta K & Potrykus 1 (1990) Genetically engineered fertile Indica-rice recovered from protoplasts. Bio/Technology 8: 736-740.
de Framond A J (1991) A metallothionein-like gene from maize (Zea mays). Cloning and characterization. FEBS Lett 290: 103-6.
Dillin A (2003) The specifics of small interfering RNA specificity. Proc Natl Acad Sci USA 100: 6289-6291.
Dong, J J, Teng, W M, Buchholz W G & Hall T C (1996). Agrobacterium-mediated transformation of javanica rice. Mol Breeding 2: 267-276.
Ebel S, Lane A N & Brown T (1992) Thermodynamic stability and solution conformation of tandem G.A mismatches in RNA and RNA.DNA hybrid duplexes. Biochem 31: 12083-12086.
Elbashir S M, Harborth J, Lendeckel W, Yalcin A, Weber K & Tuschl T (2001a) Duplexes of 21-nucleotide RNAs mediate RNA interference in cultured mammalian cells. Nature 411: 494-498.
Elbashir S M, Lendeckel W & Tuschl T (2001 b) RNA interference is mediated by 21- and 22-nucleotide RNAs. Genes Dev 15: 188-200.
Elbashir S M, Martinez J, Patkaniowska A, Lendeckel W & Tuschl T (2001 c) Functional anatomy of siRNAs for mediating efficient RNAi in Drosophila melanogaster embryo lysate. EMBO J. 20: 6877-88.
Elbashir S M, Harborth J, Weber K & Tucchl T (2002) Analysis of gene function in somatic mammalian cells using small interfering DNAs, Methods 26: 199-213
EP 0 292 435
EP 0 332 104
EP 0 332 581
EP 0 342 926
EP 0 359 472
EP 0 385 962
EP 0 392 225
EP 0 452 269
Fire A, Xu S, Montgomery M K, Kostas S A, Driver S E & Mello C C (1998) Chromatin silencing and the maintenance of a functional germline in Caenorhabditis elegans. Nature 391: 806-811.
Fire A (1999) RNA-triggered gene silencing. Trends Genet 15: 358-363.
Firek S, Draper J, Owen M R, Gandecha A, Cockburn B & Whitelam G C (1993) Secretion of a functional single-chain Fv protein in transgenic tobacco plants and cell suspension cultures. Plant Mol Biol 22: 129-142.
Freier S M, Kierzek R, Jaeger J A, Sugimoto N, Caruthers M H, Neilson T & Turner D H (1986) Improved Free-Energy Parameters for Predictions of RNA Duplex Stability. Proc Natl Acad Sci USA 83: 9373-9377.
Fromm M E, Morrish F, Armstrong C, Williams R, Thomas J & Klein T M. (1990) Inheritance and expression of chimeric genes in the progeny of transgenic maize plants. Biotechnology (NY) 8: 833-839.
Gallie D R, Sleat D E, Watts J W, Turner P C & Wilson T M (1987) A comparison of eukaryotic viral 5′-leader sequences as enhancers of mRNA expression in vivo. Nucl Acids Res 15: 8693-8711.
Glover D M & Hames B D (1995) DNA Cloning: A Practical Approach, 2nd ed. IRL Press at Oxford University Press, Oxford; New York.
Goeddel (1990) Gene Expression Technology. Methods in Enzymology, Volume 185, Academic Press, San Diego, Calif., United States of America.
Gritz L & Davies J (1983) Plasmid-encoded hygromycin B resistance: the sequence of hygromycin B phosphotransferase gene and its expression in Escherichia coli and Saccharomyces cerevisiae. Gene 25: 179-188.
Gordon-Kamm W J, Spencer T M, Mangano M L, Adams T R, Daines R J, Start W G, O'Brien J V, Chambers S A, Adams W R Jr, Willetts N G, Rice T B, Mackey C J, Krueger R W, Kausch A P & Lemaux P G (1990) Transformation of Maize Cells and Regeneration of Fertile Transgenic Plants. Plant Cell 2: 603-618.
Hammond S M, Bernstein E, Beach D & Hannon G J (2000) An RNA-directed nuclease mediates post-transcriptional gene silencing in Drosophila cells. Nature 404: 293-296.
Henikoff S & Henikoff J G (1992) Amino Acid Substitution Matrices from Protein Blocks. Proc Natl Acad Sci U SA 89: 10915-10919.
Hiei Y, Komari T & Kubo T (1997) Transformation of rice mediated by Agrobacterium tumefaciens. Plant Mol Biol 35: 205-18.
Hiei Y, Ohta S, Komari T & KumashiroY (1994) Efficient transformation of rice (Oryza sativa L.) mediated by Agrobacterium and sequence analysis of the boundaries of the T-DNA. Plant J 6(2): 271-282.
Höfgen R & Willmitzer L (1988) Storage of competent cells for Agrobacterium transformation. Nucl Acids Res 16: 9877.
Hudspeth R L & Grula J W (1989) Structure and expression of the maize gene encoding the phosphoenolpyruvate carboxylase isozyme involved in C4 photosynthesis. Plant Molec Biol 12: 579-589.
Hu W-J, Kawaoka A, Tsai C-J, Lung J, Osakabe K, Ebinuma H & Chiang V L (1998) Compartmentalized expression of two structurally and functionally distinct 4-coumarate:CoA ligase genes in aspen (Populus tremuloides). Proc Natl Acad Sci USA 95: 5407-5412.
Hutvagner G, Mlynarova L & Nap J-P (2000) Detailed characterization of the posttranscriptional gene-silencing-related small RNA in a GUS gene-silenced tobacco. RNA 6: 1445-1454.
Jefferson R A, Kavanagh T A & Bevan M W (1987) GUS fusions: beta-glucuronidase as a sensitive and versatile gene fusion marker in higher plants. EMBO J. 6: 3901-3907.
Karlin S & Altschul S F (1993) Applications and Statistics for Multiple High-Scoring Segments in Molecular Sequences. Proc Natl Acad Sci USA 90: 5873-5877.
Klein T M, Wolf E D, Wu R & Sanford J C (1987) High velocity microprojectiles for delivering nucleic acids into living cells. Nature 327: 70-73.
Koehler S M & Ho T M (1990) Hormonal regulation, processing, and secretion of cysteine proteinases in barley aleurone layers. Plant Cell 2: 769-783.
Koziel M G, Beland G L, Bowman C, Carozzi N B, Crenshaw R, Crossland L, Dawson J, Desai N, Hill M, Kadwell S, Launis L, Lewis K, Maddox D, McPherson K, Meghji M R, Merlin E, Rhodes R, Warren G W, Wright M & Evola S V (1993) Field performance of elite transgenic maize plants expressing an insecticidal protein derived from Bacillus thuringiensis. Bio/Technology 11: 194-200.
Lee N S, Dohjima T, Bauer G, Li H, Li M-J, Ehsani A, Salvaterra P & Rossi J (2002) Expression of small interfering RNAs targeted against HIV-1 rev transcripts in human cells. Nature Biotechnol. 20: 500-505.
Logemann J, Lipphardt S, Lorz H, Hauser I, Willmitzer L & Schell J (1989) 5′ upstream sequences from the wun1 gene are responsible for gene activation by wounding in transgenic plants. Plant Cell 1: 151-158.
Mayo O (1987) The Theory of Plant Breeding, Second Edition, Clarendon Press, New York, N.Y., United States of America.
McBride K E, Schaaf D J, Daley M & Stalker D M (1994) Controlled expression of plastid transgenes in plants based on a nuclear DNA-encoded and plastid-targeted T7 RNA polymerase. Proc Natl Acad Sci USA 91: 7301-7305.
McBride K E & Summerfelt K R (1990) Improved binary vectors for Agrobacterium-mediated plant transformation. Plant Mol Biol 14: 269-276.
McElroy D, Blowers A D, Jenes B & Wu R (1991) Construction of expression vectors based on the rice actin 1 (Act1) 5′ region for use in monocot transformation. Mol. Gen. Genet. 231: 150-160.
McElroy D, Zhang W, Cao J & Wu R (1990) Isolation of an efficient actin promoter for use in rice transformation. Plant Cell 2: 163-71.
McManus M T & Sharp P A (2002) Gene silencing in mammals by small interfering RNAs. Nat Rev Genet 3: 737-747.
   Messing J & Vieira J (1982) A new pair of M13 vectors for selecting either DNA strand of double-digest restriction fragments. Gene 19: 259-268.
Miyagishi M & Taira K (2002) U6-promoter-driven siRNAs with four uridine 3′ overhangs efficiently suppress targeted gene expression in mammalian cells. Nature Biotechnol. 20: 497-500.
Murashige T & Skoog F (1962) A revised medium for rapid growth and bioassays with tobacco tissue cultures. Physiol Plant 15: 473-497.
Needleman S B & Wunsch C D (1970) A General Method Applicable to the Search for Similarities in the Amino Acid Sequence of Two Proteins. J Mol Biol 48: 443-453.
Negrotto D, Jolley M, Beer S, Wenck A R & Hansen G (2000)The use of phosphomannose-isomerase as a selection marker to recover transgenic maize plant (Zea may L.) via Agrobacterium transformation. Plant Cell Reports 19: 798-803.
Nykanen A, Haley B & Zamore P D (2001) ATP requirements and small interfering RNA structure in the RNA interference pathway. Cell 107: 309-321.
Paddison P J, Caudy M, Bernstein E, Hannon G J & Conklin D S (2002) Short hairpin RNAs (shRNAs) induce sequence-specific silencing in mammalian cells. Genes & Dev. 16: 948-958.
Paszkowski J, Shillito R D, Saul M, Mandak V, Hohn T, Hohn B & Potrykus I (1984) Direct gene transfer to plants. EMBO J. 3: 2717-2722.
Paul C P, Good P D, Winer I & Engelke D R (2002) Effective expression of small interfering RNA in human cells. Nature Biotechnol. 20: 505-508.
PCT International Publication WO 93/07278
PCT International Publication WO 93/21335
PCT International Publication WO 94/00977
PCT International Publication WO 95/19431
PCT International Publication WO 96/06166
PCT International Publication WO 97/32011
PCT International Publication WO 98/54311
PCT International Publication WO 99/07409
PCT International Publication WO 99/32619
PCT International Publication WO 99/53050
PCT International Publication WO 99/61631
PCT International Publication WO 00/01846
PCT International Publication WO 00/44895
PCT International Publication WO 00/44914
PCT International Publication WO 00/63364
PCT International Publication WO 01/04313
PCT International Publication WO 01/29058
PCT International Publication WO 01/36646
PCT International Publication WO 01/68836
PCT International Publication WO 01/75164
PCT International Publication WO 01/92513
PCT International Publication WO 02/44321
PCT International Publication WO 02/055692
PCT International Publication WO 02/055693
Pearson W R & Lipman D J (1988) Improved Tools for Biological Sequence Comparison. Proc Natl Acad Sci USA 85: 2444-2448.
Potrykus I, Paszkowski J, Saul M W, Petruska J & Shillito R D (1985) Molecular and general genetics of a hybrid foreign gene introduced into tobacco by direct gene transfer. Mol Gen Genet 199: 169-177.
Reed J N, Privalle L S, Powell M J, Meghji M, Dawson J, Dunder E M, Suttie J, Wenck A, Launis K, Kramer C, Chang Y, Hansen G & Wright M (2001) Phosphomannose isomerase: An efficient selectable marker for plant transformation. In Vitro Cell Dev Biol-Plant 37: 127-132.
Reich T J, Iyer V N & Miki B L (1986) Efficient transformation of alfalfa protoplasts by the intranuclear microinjection of Ti-plasmids. Bio/Technology 4: 1001-1004.
Rogers J C, Dean D & Heck G R (1985) Aleurain: a barley thiol protease closely related to mammalian cathepsin H. Proc. Natl. Acad. Sci. USA 82: 6512-6516.
Rohrmeier T & Lehle L (1993) WIP1, a wound-inducible gene from maize with homology to Bowman-Birk proteinase inhibitors. Plant Mol Biol 22: 783-792.
Rothstein S J, Lahners K N, Lotstein R J, Carozzi N B, Jayne S M &Rice D A (1987) Promoter cassettes, antibiotic-resistance genes, and vectors for plant transformation. Gene 53: 153-161.
Sambrook J & Russell D W (2001) Molecular Cloning: A Laboratory Manual, 3rd ed. Cold Spring Harbor Laboratory Press, Cold Spring Harbor, N.Y.
Scharfmann R, Axelrod J H & Verma I M (1991) Long-Term in Vivo Expression of Retrovirus-Mediated Gene Transfer in Mouse Fibroblast Implants. Proc Natl Acad Sci USA 88: 4626-4630.
Schmidhauser & Helinski (1985) J Bacteriol 164: 446-455
Schocher R J, Shillito R D, Saul M W, Paszkowski J & Potrykus 1 (1986) Co-transformation of foreign genes into plants. Bio/Technology 4: 1093-1096.
Shimamoto K, Terada R, Izawa T & Fujimoto H (1989) Fertile transgenic rice plants regenerated from transformed protoplasts. Nature 338: 274-276.
Shinshi H, Neuhas J M, Ryals J & Meins F Jr (1990) Structure of a tobacco endochitinase gene: evidence that different chitinase genes can arise by transposition of sequences encoding a cysteine-rich domain. Plant Mol Biol 14: 357-368.
Silhavy T J, Berman M L, Enquist L W & Cold Spring Harbor Laboratory. (1984) Experiments with Gene Fusions. Cold Spring Harbor Laboratory, Cold Spring Harbor, N.Y.
Singh D P (1986) Breeding for Resistance to Diseases and Insect Pests, Springer-Verlag, New York, N.Y., United States of America.
Skuzeski J M, Nichols L M & Gesteland R F (1990) Analysis of leaky viral translation termination codons in vivo by transient expression of improved beta-glucuronidase vectors. Plant Mol Biol 15: 65-79.
Smith T F & Waterman M (1981) Comparison of Biosequences. Adv Appl Math 2: 482-489.
Spencer T M, Gordon-Kamm W J, Daines R J, Start W & Lemaux P (1990). Theor Appl Genet 79: 625-631.
Sui G, Soohoo C, Affar E-B, Gay F, Shi Y, Forrester W C & Shi Y (2002) A DNA vector-based RNAi technology to suppress gene expression in mammalian cells. Proc Natl Acad Sci USA 99: 5515-5520.
Svab Z, Hajdukiewicz P & Maliga P (1990) Stable transformation of plastids in higher plants. Proc Natl Acad Sci USA 87: 8526-8530.
Svab Z & Maliga P (1993) High-frequency plastid transformation in tobacco by selection for a chimeric aadA gene. Proc Natl Acad Sci USA 90: 913-917.
Taylor M, Vasil V & Vasil I K (1993) Enhanced gus gene expression in cereal grass cell suspensions and immature embryos using the maize ubiquitin-based plasmid pAHC25. Plant Cell Rep 12: 491-495.
Thompson C J, Mowa N R, Tizard R, Crameri R, Davies J E, Lauwereys M & Botterman J (1987) Characterization of the herbicide-resistance gene bar from Streptomyces hygroscopicus. EMBO J. 6: 2519-2523.
Tibanyenda N, De Bruin S H, Haasnoot C A, van der Marel G A, van Boom J H & Hilbers C W (1984) The effect of single base-pair mismatches on the duplex stability of d(T-A-T-T-A-A-T-A-T-C-A-A-G-T-T-G). d(C-A-A-C-T-T-G-A-T-A-T-T-A-A-T-A). Eur J Biochem 139: 19-27.
Tijssen P (1993) Laboratory Techniques in Biochemistry and Molecular Biology-Hybridization with Nucleic Acid Probes. Elsevier, New York, United States of America.
Turner D H, Sugimoto N, Jaeger J A, Longfellow C E, Freier S M & Kierzek R (1987) Improved parameters for prediction of RNA structure. Cold Spring Harb Symp Quant Biol LII: 123-133.
Uknes S, Dincher S, Friedrich L, Negrotto D, Williams S, Thompson-Taylor H, Potter S, Ward E & Ryals J (1993) Regulation of pathogenesis-related protein-1 a gene expression in tobacco. Plant Cell 5: 159-169.
Uknes S, Mauch-Mani B, Moyer M, Potter S, Williams S, Dincher S, Chandler D, Slusarenko A, Ward E & Ryals J (1992) Acquired resistance in Arabidopsis. Plant Cell 4: 645-656.
Unger E A, Hand J M, Cashmore A R & Vasconcelos A C (1989) Isolation of a cDNA encoding mitochondrial citrate synthase from Arabidopsis thaliana. Plant Mol Biol 13: 411-418.
U.S. Patent Application No. 20030108923
U.S. Patent Application No. 20030104401
U.S. Patent Application No. 20030068821
U.S. Pat. No. 4,554,101
U.S. Pat. No. 4,940,935
U.S. Pat. No. 4,945,050
U.S. Pat. No. 4,987,071
U.S. Pat. No. 5,036,006
U.S. Pat. No. 5,100,792
U.S. Pat. No. 5,188,642
U.S. Pat. No. 5,270,163
U.S. Pat. No. 5,466,785
U.S. Pat. No. 5,501,967
U.S. Pat. No. 5,523,311
U.S. Pat. No. 5,591,616
U.S. Pat. No. 5,614,395
U.S. Pat. No. 5,639,949
U.S. Pat. No. 5,767,378
U.S. Pat. No. 5,994,629
U.S. Pat. No. 6,369,298
U.S. Pat. No. 6,455,762
Van den Broeck G, Timko M P, Kausch A P, Cashmore A R, Van Montagu M & Herrera-Estrella L (1985) Targeting of a foreign protein to chloroplasts by fusion to the transit peptide from the small subunit of ribulose 1,5-bisphosphate carboxylase. Nature 313: 358-363.
Vasil V, Castillo A M, Fromm M E & Vasil I K (1992) Herbicide resistant fertile transgenic wheat plants obtained by microprojectile bombardment of regenerable embryogenic callus. Bio/Technology 10: 667-674.
Vasil V, Srivastava V, Castillo A M, Fromm M R & Vasil I K (1993) Rapid production of transgenic plants by direct bombardment of cultured immature embryos. Bio/Technology 11: 1553-1558.
Warner S A, Scott R, Draper J (1993) Isolation of an asparagus intracellular PR gene (AoPR1) wound-responsive promoter by the inverse polymerase chain reaction and its characterization in transgenic tobacco. Plant J 3: 191-201.
Wasmann C C et al. (1986) Mol Gen Genet 205: 446-453.
Waterhouse P M, Wang M B & Lough T (2001) Gene silencing as an adaptive defence against viruses. Nature 411: 834-842.
Weeks J T, Anderson O D & Blechl A E (1993) Rapid Production of Multiple Independent Lines of Fertile Transgenic Wheat (Triticum aestivum). Plant Physiol 102: 1077-1084.
Welsh J R (1981) Fundamentals of Plant Genetics and Breedinq, John Wiley & Sons, New York, N.Y., United States of America.
White J, Chang S Y & Bibb M J (1990) A cassette containing the bar gene of Streptomyces hygroscopicus: a selectable marker for plant transformation. Nucl Acids Res 18: 1062.
Wianny F & Zernicka-Goetz M (1999) Specific interference with gene function by double-stranded RNA in early mouse development. Nature Cell Biol 2: 70-75.
Williams R S, Thomas J A, Fina M, German Z & Benjamin I J (1993) Human Heat Shock Protein 70 (Hsp70) Protects Murine Cells from Injury During Metabolic Stress. J Clin Invest 92: 503-508.
Wood D R (ed.) (1983) Crop Breeding, American Society of Agronomy, Madison, Wis., United States of America.
Wricke G & Weber W E (1986) Quantitative Genetics and Selection Plant Breeding, Walter de Gruyter and Co., Berlin, Germany.
Xu D, McElroy D, Thornburg RW & Wu R (1993) Systemic induction of a potato pin2 promoter by wounding, methyl jasmonate, and abscisic acid in transgenic rice plants. Plant Mol Biol 22: 573-588.
Yu J-Y, DeRuiter SL & Turner DL (2002) RNA interference by expression of short-interfering RNAs and hairpin RNAs in mammalian cells. Proc Natl Acad Sci USA 99: 6047-6052.
Zhang H M, Yang H, Rech E L, Golds T J, Davis A S, Mulligan B J, Cocking E C & Davey M R (1988) Transgenic rice plants produced by electroporation-mediated plasmid uptake into protoplasts. Plant Cell Reports 7: 379-384.

It will be understood that various details of the presently disclosed subject matter can be changed without departing from the scope of the presently disclosed subject matter. Furthermore, the foregoing description is for the purpose of illustration only, and not for the purpose of limitation.

1. A method for stably modulating expression of a plant gene, the method comprising:
(a) providing a vector encoding a short interfering RNA (siRNA) targeted to the plant gene; and
(b) transforming a plant cell with the vector, whereby stable expression of the siRNA in the plant cell is provided.

2. The method of claim 1, wherein the vector is an Agrobacterium binary vector.

3. The method of claim 1, wherein the vector comprises:
(a) a promoter operatively linked to a nucleic acid molecule encoding the siRNA molecule; and
(b) a transcription termination sequence.

4. The method of claim 3, wherein the vector is an Agrobacterium binary vector.

5. The method of claim 3, wherein the promoter is a DNA-dependent RNA polymerase III promoter.

6. The method of claim 5, wherein the promoter is selected from the group consisting of an RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, and a tRNA gene promoter, or a functional derivative thereof.

7. The method of claim 6, wherein the Arabidopsis thaliana 7SL RNA gene promoter comprises the sequence presented in SEQ ID NO: 3.

8. The method of claim 3, wherein the nucleic acid sequence encoding the short interfering RNA (siRNA) molecule comprises a sense region, an antisense region, and a loop region, positioned in relation to each other such that upon transcription, the resulting RNA molecule is capable of forming a hairpin structure via intramolecular hybridization of the sense strand and the antisense strand.

9. The method of claim 1, wherein the plant is a dicot.

10. The method of claim 1, wherein the plant is a monocot.

11. The method of claim 1, wherein the plant is a tree.

12. The method of claim 11, wherein the tree is an angiosperm.

13. The method of claim 11, wherein the tree is a gymnosperm.

14. The method of claim 1, wherein the plant is selected from the group consisting of Arabidopsis, poplar, aspen, and tobacco.

15. The method of claim 1, wherein the stable expression of the short interfering RNA (siRNA) in the plant occurs in a location or tissue selected from the group consisting of epidermis, root, vascular tissue, xylem, meristem, cambium, cortex, pith, leaf, flower, seed, and combinations thereof.

16. A vector for stably expressing a short interfering RNA (siRNA) molecule in a plant, the vector comprising:
(a) a promoter operatively linked to a nucleic acid molecule encoding the siRNA molecule; and
(b) a transcription termination sequence.

17. The vector of claim 16, wherein the vector is an Agrobacterium binary vector.

18. The vector of claim 16, wherein the promoter is a DNA-dependent RNA polymerase III promoter.

19. The vector of claim 18, wherein the promoter is selected from the group consisting of RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, and a tRNA gene promoter, or a functional derivative thereof.

20. The vector of claim 19, wherein the Arabidopsis thaliana SL7 RNA gene promoter comprises the sequence presented in SEQ ID NO: 3.

21. The vector of claim 16, wherein the nucleic acid sequence encoding the short interfering RNA (siRNA) molecule comprises a sense region, an antisense region, and a loop region, positioned in relation to each other such that upon transcription, the resulting RNA molecule is capable of forming a hairpin structure via intramolecular hybridization of the sense strand and the antisense strand.

22. A kit comprising the vector of claim 16 and at least one reagent for introducing the vector of claim 15 into a plant cell.

23. The kit of claim 22, further comprising instructions for introducing the vector into a plant cell.

24. A plant cell comprising the vector of claim 16.

25. A transgenic plant comprising the vector of claim 16.

26. Transgenic seed or progeny from the transgenic plant of claim 25.

27. A method for enhancing the expression of a gene in a plant cell, the method comprising introducing into the plant cell a vector encoding a short interfering RNA (siRNA) molecule corresponding to at least a subsequence of the gene.

28. The method of claim 27, wherein the gene is selected from the group consisting of coniferaldehyde-5-hydroxylase (Cald5H), a lignin-related gene, a cellulose-related gene, a hormone-related gene, a disease-related gene, a stress-related gene, and a transcription factor gene.

29. The method of claim 28, wherein the lignin-related gene is selected from the group consisting of sinapyl alcohol dehydrogenase (SAD), cinnamyl alcohol dehydrogenase (CAD), 4-coumarate:CoA ligase (4CL), cinnamoyl CoA O-methyltransferase (CCOAOMT; also referred to as CCOMT), caffeate O-methyltransferase (COMT), ferulate-5-hydroxylase (F5H), cinnamate-4-hydroxylase (C4H), p-coumarate-3-hydroxylase (C3H), cinnamoyl CoA reductase (CCR), and phenylalanine ammonia lyase (PAL).

30. The method of claim 28, wherein the cellulose-related gene is selected from the group consisting of cellulose synthase (CeS or CESA), cellulose synthase-like (CSL), glucosidase, glucan synthase, Korrigan endocellulase, callose synthase, and sucrose synthase.

31. The method of claim 28, wherein the hormone-related gene is selected from the group consisting of isopentyl transferase (ipt), gibberellic acid (GA) oxidase, auxin (AUX), auxin-responsive and auxin-induced genes, and members of the ROL gene family.

32. A method for enhancing the expression of a gene in a plant cell, the method comprising introducing into the plant cell a vector encoding a short interfering RNA (siRNA) molecule comprising a sequence that hybridizes to a nucleic acid molecule encoding a repressor of the gene, thereby resulting in downregulation of expression of the repressor.

33. A method for stably inhibiting expression of a gene in a plant cell, the method comprising introducing a vector encoding an siRNA into the cell in an amount sufficient to inhibit expression of the gene, wherein the siRNA comprises a ribonucleotide sequence which corresponds to at least 15 contiguous nucleotides of a coding strand of the gene.

34. The method of claim 33, wherein the gene is selected from the group consisting of coniferaldehyde-5-hydroxylase (Cald5H), a lignin-related gene, a cellulose-related gene, a hormone-related gene, a disease-related gene, a stress-related gene, and a transcription factor gene.

35. The method of claim 34, wherein the lignin-related gene is selected from the group consisting of sinapyl alcohol dehydrogenase (SAD), cinnamyl alcohol dehydrogenase (CAD), 4-coumarate:CoA ligase (4CL), cinnamoyl CoA O-methyltransferase (CCOAOMT; also referred to as CCOMT), caffeate O-methyltransferase (COMT), ferulate-5-hydroxylase (F5H), cinnamate-4-hydroxylase (C4H), p-coumarate-3-hydroxylase (C3H), cinnamoyl CoA reductase (CCR), and phenylalanine ammonia lyase (PAL).

36. The method of claim 34, wherein the cellulose-related gene is selected from the group consisting of cellulose synthase (CeS or CESA), cellulose synthase-like (CSL), glucosidase, glucan synthase, Korrigan endocellulase, callose synthase, and sucrose synthase.

37. The method of claim 34, wherein the hormone-related gene is selected from the group consisting of isopentyl transferase (ipt), gibberellic acid (GA) oxidase, auxin (AUX), auxin-responsive and auxin-induced genes, and members of the ROL gene family.

38. The method of claim 33, wherein the siRNA comprises a double-stranded region comprising a first strand comprising a ribonucleotide sequence that corresponds to a coding strand of the gene and a second strand comprising a ribonucleotide sequence that is complementary to the first strand, and wherein the first strand and the second strand hybridize to each other to form the double-stranded region.

39. The method of claim 38, wherein the double stranded region is at least 15 basepairs in length.

40. The method of claim 39, wherein the double stranded region is between 15 and 50 basepairs in length.

41. The method of claim 40, wherein the double stranded region is between 15 and 30 basepairs in length.

42. The method of claim 41, wherein a length of the double stranded region is selected from the group consisting of 19, 20, 21, 22, 23, 24, 25, and 26 basepairs.

43. The method of claim 42, wherein the length of the double stranded region is 19 basepairs.

44. The method of claim 33, wherein the expression of the gene is inhibited by at least 10%.

45. The method of claim 33, wherein the RNA comprises one strand that forms a double-stranded region of at least 19 basepairs by intramolecular self-hybridization.

46. An expression vector encoding a short interfering RNA (siRNA) molecule that stably down regulates expression of a plant gene by RNA interference.

47. The expression vector of claim 46, wherein the short interfering RNA (siRNA) molecule comprises a sense region and an antisense region, and wherein the antisense region comprises a nucleic acid sequence complementary to an RNA sequence encoded by the plant gene and the sense region comprises a nucleic acid sequence complementary to the antisense region.

48. The expression vector of claim 47, wherein the short interfering RNA (siRNA) molecule is assembled from two nucleic acid fragments, wherein one fragment comprises a sense region and the other fragment comprises an antisense region of the siRNA molecule.

49. The expression vector of claim 48, wherein the sense region and antisense region are covalently connected via a linker molecule.

50. The expression vector of claim 49, wherein the linker molecule is a polynucleotide linker.

51. The expression vector of claim 50, wherein the polynucleotide linker comprises from 5 to 9 nucleotides.

52. The expression vector of claim 50, wherein the short interfering RNA (siRNA) molecule is formed by intramolecular self-hybridization of the sense region and the antisense region to produce a double-stranded molecule, and the double-stranded molecule comprises 3′-terminal overhang of at least 2 nucleotides.

53. The expression vector of claim 52, wherein the 3′-terminal overhang comprises from 2 to 8 nucleotides.

54. The expression vector of claim 46, wherein the antisense region is complementary to a ribonucleic acid (RNA) transcribed from a gene selected from the group consisting of coniferaldehyde-5-hydroxylase (Cald5H), a lignin-related gene, a cellulose-related gene, a hormone-related gene, a disease-related gene, a stress-related gene, and a transcription factor gene.

55. The expression vector of claim 54, wherein the lignin-related gene is selected from the group consisting of sinapyl alcohol dehydrogenase (SAD), cinnamyl alcohol dehydrogenase (CAD), 4-coumarate:CoA ligase (4CL), cinnamoyl CoA O-methyltransferase (CCOAOMT; also referred to as CCOMT), caffeate O-methyltransferase (COMT), ferulate-5-hydroxylase (F5H), cinnamate-4-hydroxylase (C4H), p-coumarate-3-hydroxylase (C3H), cinnamoyl CoA reductase (CCR), and phenylalanine ammonia lyase (PAL).

56. The expression vector of claim 54, wherein the cellulose-related gene is selected from the group consisting of cellulose synthase (CeS or CESA), cellulose synthase-like (CSL), glucosidase, glucan synthase, Korrigan endocellulase, callose synthase, and sucrose synthase.

57. The expression vector of claim 54, wherein the hormone-related gene is selected from the group consisting of isopentyl transferase (ipt), gibberellic acid (GA) oxidase, auxin (AUX), auxin-responsive and auxin-induced genes, and members of the ROL gene family.

58. The expression vector of claim 46, wherein the short interfering RNA (siRNA) molecule comprises a sense region and an antisense region and wherein the antisense region comprises a nucleic acid sequence complementary to an RNA sequence transcribed from a gene selected from the group consisting of coniferaldehyde-5-hydroxylase (Cald5H), a lignin-related gene, a cellulose-related gene, a hormone-related gene, a disease-related gene, a stress-related gene, and a transcription factor gene, and the sense region comprises a nucleic acid sequence complementary to the antisense region.

59. The expression vector of claim 58, wherein the lignin-related gene is selected from the group consisting of sinapyl alcohol dehydrogenase (SAD), cinnamyl alcohol dehydrogenase (CAD), 4-coumarate:CoA ligase (4CL), cinnamoyl CoA O-methyltransferase (CCOAOMT; also referred to as CCOMT), caffeate O-methyltransferase (COMT), ferulate-5-hydroxylase (F5H), cinnamate-4-hydroxylase (C4H), p-coumarate-3-hydroxylase (C3H), cinnamoyl CoA reductase (CCR), and phenylalanine ammonia lyase (PAL).

60. The expression vector of claim 58, wherein the cellulose-related gene is selected from the group consisting of cellulose synthase (CeS or CESA), cellulose synthase-like (CSL), glucosidase, glucan synthase, Korrigan endocellulase, callose synthase, and sucrose synthase.

61. The expression vector of claim 58, wherein the hormone-related gene is selected from the group consisting of isopentyl transferase (ipt), gibberellic acid (GA) oxidase, auxin (AUX), auxin-responsive and auxin-induced genes, and members of the ROL gene family.

62. The expression vector of claim 46, wherein the short interfering RNA (siRNA) molecule comprises a single strand having complementary sense and antisense regions.

63. A plant cell comprising an expression vector of claim 46.

64. The plant cell of claim 63, wherein the plant cell is from a plant selected from the group consisting of poplar, pine, eucalyptus, sweetgum, other tree species, tobacco, Arabidopsis, rice, corn, wheat, cotton, potato, and cucumber.

65. A plasmid vector encoding a short interfering RNA (siRNA) molecule that stably down regulates expression of a plant gene by RNA interference.

66. The plasmid vector of claim 65, wherein the short interfering RNA (siRNA) molecule comprises a sense region and an antisense region, and wherein the antisense region comprises a nucleic acid sequence complementary to an RNA sequence encoded by the plant gene and the sense region comprises a nucleic acid sequence complementary to the antisense region.

67. A vector for the stable expression of a short interfering RNA (siRNA) in a plant, wherein the vector comprises a promoter for expressing the siRNA, a transcription termination sequence, and a cloning site between the promoter and the transcription termination sequence into which a nucleic acid molecule encoding the siRNA can be cloned.

68. The vector of claim 67, wherein the promoter is a DNA-dependent RNA polymerase III promoter.

69. The vector of claim 68, wherein the promoter is selected from the group consisting of RNA polymerase III H1 promoter, an Arabidopsis thaliana 7SL RNA promoter, an RNA polymerase III 5S promoter, an RNA polymerase III U6 promoter, an adenovirus VA1 promoter, a Vault promoter, a telomerase RNA promoter, and a tRNA gene promoter, or a functional derivative thereof.

70. The vector of claim 69, wherein the Arabidopsis thaliana 7SL RNA gene promoter comprises SEQ ID NO: 3.

71. The vector of claim 67, wherein the vector is a plasmid vector.

72. The vector of claim 71, wherein the vector further comprises a selectable marker.

73. The vector of claim 71, wherein the vector further comprises a cloning site comprising recognition sequences for at least two restriction enzymes that are not present elsewhere in the plasmid vector.

74. A method for stably modulating expression of a gene in a plant, the method comprising:
(a) transforming a plurality of plant cells to create a plurality of transformed plant cells, wherein the transformed plants cells have been transformed with a vector comprising a nucleic acid sequence encoding a short interfering RNA (siRNA) operatively linked to a promoter and a transcription termination sequence;
(b) growing the transformed plant cells under conditions sufficient to select for those transformed plant cells that have integrated the vector into their genomes;
(c) screening the plurality of transformed plant cells for expression of the siRNA encoded by the vector;
(d) selecting a plant cell that expresses the siRNA; and
(e) regenerating the plant from the plant cell that expresses the siRNA, whereby expression of the gene in the plant is stably modulated.

75. The method of claim 74, wherein the nucleic acid sequence encoding the short interfering RNA (siRNA) comprises:
(a) a sense region;
(b) an antisense region; and
(c) a loop region,
wherein the sense, antisense, and loop regions are positioned in relation to each other such that upon transcription, the resulting RNA molecule is capable of forming a hairpin structure via intramolecular hybridization of the sense strand and the antisense strand.

76. The method of claim 74, wherein the vector is an Agrobacterium binary vector further comprising a nucleic acid encoding a selectable marker operatively linked to a promoter.
